(19) United States
(12) Patent Application Publication
(10) Pub. No.: US 2021/0213121 A1
(43) Pub. Date: Jul. 15, 2021

(54) NEOANTIGEN TARGETING DNA VACCINE FOR COMBINATION THERAPY

(71) Applicant: VAXIMM AG, Basel (DE)

(72) Inventor: Heinz Lubenau, Neustadt an der Weinstrasse (DE)

(21) Appl. No.: 17/273,590

(22) PCT Filed: Sep. 4, 2019
(86) PCT No.: PCT/EP2019/073564

§ 371 Date: Mar. 4, 2021

(30) Foreign Application Priority Data
Sep. 5, 2018 (EP) 18192782.3
Jan. 3, 2019 (EP) 19150251.7

(51) Int. Cl. A61K 39/00; A61K 45/06; A61K 9/00; A61P 35/00

(52) U.S. Cl. CPC A61K 39/001182; A61K 45/06; A61K 9/0053; A61P 35/00; A61K 39/001109; A61K 2039/70; A61K 39/001153; A61K 2039/545; A61K 2039/54; A61K 2039/523; A61K 39/001168

(57) ABSTRACT

The present invention relates to a Salmonella typhi Ty21a strain comprising a DNA molecule comprising at least one eukaryotic expression cassette encoding at least one polypeptide comprising five or more neoantigens and at least one engineered T cell, NKT cell or NK cell comprising at least one tumor antigen binding cell surface receptor for combined use in the treatment of a solid tumor in a subject.

### FIELD OF THE INVENTION

The present invention relates to a Salmonella typhi Ty21a strain comprising a DNA molecule comprising at least one eukaryotic expression cassette encoding at least one polypeptide comprising five or more neoantigens and at least one engineered T cell, NKT cell or NK cell comprising at least one tumor antigen binding cell surface receptor for combined use in the treatment of a solid tumor in a subject.

### BACKGROUND OF THE INVENTION

The finding that tumors can be immunogenic has led to the development of a number of cancer immunotherapies designed to employ the immune system to selectively eliminate malignant cells while sparing normal tissue. However, survival benefits from vaccination against tumor antigens alone remain modest. Anti-cancer vaccines face numerous challenges, one of them being the immunosuppressive microenvironment. The abnormal tumor vasculature creates a hypoxic microenvironment that polarizes inflammatory cells toward immune suppression. Moreover, tumors systemically alter immune cells' proliferation, differentiation, and function via secretion of growth factors and cytokines.

For cure of cancer, complete eradication of cancer stem cells is of crucial importance. The numerous immune escape mechanisms of human tumors remain a major challenge in cancer immunotherapy. Thus, there exists a great need for improved cancer therapy approaches, including combined cancer therapy approaches, which has not been met so far.

Recently, adoptive cell therapy of cancer with reprogrammed T cells, such as CAR-T cells and CAR-NKT cells, as well as reprogrammed NK cells (CAR-NK cells), has shown to be promising. Although in initial attempts patients affected by a variety of solid and liquid tumors were treated, the breakthrough with CAR-T cell therapy was achieved targeting B-cell hematologic tumors. Two immunotherapies with anti-CD19 modified T cells, KYMRIAH (tisagenlecleucel) and YESCARTA (axicabtagene ciloleucel), have recently been approved by the FDA. However, while CAR-T cell therapy can be effective against some blood cancers, efficacy against common solid cancers is modest, particularly durable complete responses are rare.

Whereas the main focus to date has been on improving CAR-T cells, the transfer of CARs into cell types other than conventional αβT cells, such as γδT cells, natural killer T (NKT) cells and natural killer (NK) cells, has gained importance.

Another immunotherapeutic approach that has gained interest in cancer therapy is vaccination against cancer. While there are several ways of immunizing against cancer a very promising way is the use of bacteria such as Salmonella as carrier for a DNA vaccine against a tumor antigen or stroma antigen. For example, WO 2014/005683 discloses an attenuated strain of Salmonella comprising a recombinant DNA molecule encoding a VEGF receptor protein for use in cancer immunotherapy, particularly for use in the treatment of pancreatic cancer.

Further, WO 2014/173542 and WO 2015/090584 disclose an attenuated strain of Salmonella comprising a recombinant DNA molecule encoding Wilms' tumor protein or mesothelin for use in cancer immunotherapy.

WO 2013/09189 discloses a method for growing attenuated mutant Salmonella typhi strains lacking galactose epimerase activity and harboring a recombinant DNA molecule and WO 2018/011289 discloses a fast and effective method of generating personalized cancer vaccines comprising an attenuated strain of Salmonella.

### OBJECTS OF THE INVENTION

In view of the prior art, it is an object of the present invention to provide novel cancer therapies. Such novel therapies would offer major advantages for improving the treatment options for cancer patients with solid tumors.

### SUMMARY OF THE INVENTION

In one aspect the present invention relates to a Salmonella typhi Ty21a strain comprising a DNA molecule comprising at least one eukaryotic expression cassette encoding at least one polypeptide comprising five or more neoantigens, for use in the treatment of a solid tumor in a subject, wherein the subject has been or is treated with at least one engineered T cell, NKT cell or NK cell comprising at least one tumor antigen binding cell surface receptor.

In another aspect the present invention relates to a Salmonella typhi Ty21a strain comprising a DNA molecule comprising at least one eukaryotic expression cassette encoding at least one polypeptide comprising five or more neoantigens in combination with at least one engineered T cell, NKT cell or NK cell comprising at least one tumor antigen binding cell surface receptor for use in the treatment of a solid tumor in a subject.

According to the invention, the at least five or more neoantigens are tumor specific antigens identified in the solid tumor of said subject. Preferably the five or more neoantigens comprise CD8 T cells antigens or CD8 and CD4 T cell antigens. In one embodiment the at least one polypeptide comprises 10 or more, preferably 20 or more neoantigens, preferably 30 or more neoantigens, preferably 50 or more neoantigens. In another embodiment the at least one polypeptide comprises 5 to 300 neoantigens, 10 to 300 neoantigens, 20 to 300 neoantigens, preferably 30 to 300 neoantigens, preferably 50 to 300 neoantigens. In yet another embodiment the at least one polypeptide comprises 10 to 200 neoantigens, 10 to 200 neoantigens, 20 to 200 neoantigens, preferably 30 to 200 neoantigens, preferably 50 to 200 neoantigens.

The Salmonella typhi Ty21a strain may further comprise a DNA molecule encoding at least one polypeptide comprising a tumor specific antigen and/or tumor associated antigen that is not a neoantigen, wherein said tumor specific antigen and/or tumor associated antigen that is not a neoantigen is expressed in said solid tumor, wherein the at least one polypeptide comprising a tumor specific antigen and/or tumor associated antigen that is not a neoantigen is (a) encoded by the same DNA molecule comprising the at least one eukaryotic expression cassette encoding the at least one polypeptide comprising five or more neoantigens or by a further separate DNA molecule, (b) is encoded by the at least one eukaryotic expression cassette encoding the at least one polypeptide comprising five or more neoantigens or by a further separate expression cassette; or (c) is the at least one polypeptide comprising five or more neoantigens or a further separate polypeptide.

According to the invention, the Salmonella typhi Ty21a strain may be co-administered with at least one checkpoint inhibitor. Preferably, the at least one checkpoint inhibitor is selected from a group consisting of antibodies against PD-1, PD-L1, CTLA-4, IDO, GITR, OX40, TIM-3, LAG-3, KIR, CSF1R and CD137.

The at least one engineered T cell, NKT cell or NK cell comprising at least one tumor antigen binding cell surface receptor may be a T cell, NKT cell or NK cell comprising a chimeric antigen receptor (CAR), also referred to as chimeric antigen receptor (CAR)-T cell, CAR-NKT cell or CAR-NK cell, respectively. In one embodiment, the Salmonella typhi Ty21a strain is to be administered following adoptive cell transfer of the at least one engineered T cell, NKT cell or NK cell comprising at least one tumor antigen binding cell surface receptor. Preferably, the Salmonella typhi Ty21a strain is administered about 2 weeks to 4 months, preferably 2 to 3 months, after a first adoptive cell transfer of the at least one engineered T cell, NKT cell or NK cell comprising at least one tumor antigen binding cell surface receptor.

In one embodiment prior to adoptive cell transfer of the at least one engineered T cell, NKT cell or NK cell comprising at least one tumor antigen binding cell surface receptor the subject has undergone lymphodepleting chemotherapy. In this case the subject in need of therapy is immunocompromised and lymphocyte and/or leukocyte counts need to have normalized before the Salmonella typhi Ty21a strain is to be administered.

According to the invention the Salmonella typhi Ty21a strain comprising a DNA molecule comprising at least one eukaryotic expression cassette encoding at least one polypeptide comprising five or more neoantigens may be co-administered with at least one Salmonella typhi Ty21a strain comprising a DNA molecule comprising at least one eukaryotic expression cassette encoding a tumor antigen, a tumor stroma antigen and/or a check point inhibitor antigen, preferably selected from the group consisting of human Wilms' Tumor protein (WT1), human Mesothelin (MSLN), human CEA, CMV pp65, human PD-L1, VEGFR-2 and human fibroblast activation protein (FAP). More specifically, VEGFR-2 may comprise the amino acid sequence of SEQ ID NO: 1 or an amino acid sequence that has at least 80% sequence identity with the amino acid sequence of SEQ ID NO: 1, WT1 may comprise the amino acid sequence of SEQ ID NO: 3 or an amino acid sequence that has at least 80% sequence identity with the amino acid sequence of SEQ ID NO: 3, MSLN may comprise the amino acid sequence of SEQ ID NO: 4 or an amino acid sequence that has at least 80% sequence identity with the amino acid sequence of SEQ ID NO: 4; the human CEA may comprise the amino acid sequence of SEQ ID NO: 5 or an amino acid sequence that has at least 80% sequence identity with the amino acid sequence of SEQ ID NO: 5; the CMV pp65 may comprise the amino acid sequence of SEQ ID NO: 6, 7 or 8 or an amino acid sequence that has at least 80% sequence identity with the amino acid sequence of SEQ ID NO: 6, 7 or 8; and/or the human PD-L1 may comprise the amino acid sequence of SEQ ID NO: 9 or an amino acid sequence that has at least 80% sequence identity with the amino acid sequence of SEQ ID NO: 9.

In one embodiment the Salmonella typhi Ty21a strain comprising a DNA molecule comprising at least one eukaryotic expression cassette encoding at least one polypeptide comprising five or more neoantigens is in the form of a pharmaceutical composition and may further comprise at least one pharmaceutically acceptable excipient. The pharmaceutical composition may further comprise at least one Salmonella typhi Ty21a strain comprising a DNA molecule comprising at least one eukaryotic expression cassette encoding a tumor antigen and/or a tumor stroma antigen, preferably selected from the group consisting of WT1, MSLN, CEA, CMV pp65, PD-L1, VEGFR-2 and FAP.

The solid tumor to be treated may be any solid tumor, such as colorectal cancer, pancreatic cancer, lung cancer, ovarian cancer, mesothelioma, glioblastoma, gastric cancer, hepatocellular cancer, renal cell cancer, prostate cancer, cervical cancer, breast cancer or melanoma.

A single dose of the Salmonella typhi Ty21a strain according to the invention comprises from about 10⁶ to about 10¹⁰, more particularly from about 10⁶ to about 10⁹, more particularly from about 10⁶ to about 10⁸, most particularly from about 10⁷ to about 10⁸ colony forming units (CFU). In one embodiment, the Salmonella typhi Ty21a strain according to the invention is administered two to four times in the first week, preferably 4 times in the first week, followed by single dose boosting administration every 2 to 4 weeks, particularly on day 1 and 7, preferably on day 1, 3, 5 and 7 followed by single dose boosting administrations every 2 to 4 weeks.

### SHORT DESCRIPTION OF THE FIGURES

FIG. 1: Frequency of the indicated epitope-specific CD8+ T cell population in the splenocytes of C57BL/6 mice immunized via the oral route with (A) empty vector, (B) VXMNeo1m and (C) VXM06m. Shown are % epitope-specific pentamer-positive CD8+ T cells among total CD8+ T cells for the indicated epitopes.

FIG. 2: Stability testing of drug products. Finished drug products manufactured from three constructs encoding three different target antigens (Product 1, Product 2 and Product 3) based on Salmonella typhi Ty21a delivery platform at 10⁴ (P4) 10⁵ (P5), 10⁶ (P6) or 10⁷ (P7) CFU/ml were incubated at 70° C. for the indicated time. Shown are viable cell counts (CFU/ml).

### DETAILED DESCRIPTION OF THE INVENTION

The present invention may be understood more readily by reference to the following detailed description of the invention.

In one aspect the present invention relates to a Salmonella typhi Ty21a strain comprising a DNA molecule comprising at least one eukaryotic expression cassette encoding at least one polypeptide comprising five or more neoantigens. The Salmonella typhi Ty21a strain encoding at least one polypeptide comprising five or more neoantigens may be used in the treatment of a solid tumor in a subject. Particularly, the at least five or more neoantigens are tumor specific antigens identified in the solid tumor of said subject

The invention further relates to a Salmonella typhi Ty21a strain comprising a DNA molecule comprising at least one eukaryotic expression cassette encoding at least one polypeptide comprising five or more neoantigens, for use in the treatment of a solid tumor in a subject, wherein the subject has been or is treated with at least one engineered T cell, NKT cell or NK cell comprising at least one tumor antigen binding cell surface receptor. Particularly, the at least five or more neoantigens are tumor specific antigens identified in the solid tumor of said subject. Furthermore, the at least one tumor antigen binding cell surface receptor binds to at least one tumor antigen identified to be expressed or overexpressed in the solid tumor of said subject. Thus, preferably at least one tumor antigen is identified to be expressed or overexpressed in the solid tumor of said subject and the subject has been or is treated with at least one engineered T cell, NKT cell or NK cell comprising at least one tumor antigen binding cell surface receptor targeting the at least one tumor antigen identified to be expressed or overexpressed in the solid tumor of said subject.

Moreover, the invention relates to a Salmonella typhi Ty21 a strain comprising a DNA molecule comprising at least one eukaryotic expression cassette encoding at least one polypeptide comprising five or more neoantigens in combination with at least one engineered T cell, NKT cell or NK cell comprising at least one tumor antigen binding cell surface receptor for use in the treatment of a solid tumor in a subject. Alternatively, the Salmonella typhi Ty21 a strain comprising a DNA molecule comprising at least one eukaryotic expression cassette encoding at least one polypeptide comprising five or more neoantigens is for use in the treatment of a solid tumor in a subject, in combination with at least one engineered T cell, NKT cell or NK cell comprising at least one tumor antigen binding cell surface receptor. Particularly, the at least five or more neoantigens are tumor specific antigens identified in the solid tumor of said subject. Furthermore, the at least one tumor antigen binding cell surface receptor binds to at least one tumor antigen identified to be expressed or overexpressed in the solid tumor of said subject. Thus, preferably at least one tumor antigen is identified to be express or overexpressed in the solid tumor of said subject and the subject has been or is treated with at least one engineered T cell, NKT cell or NK cell comprising at least one tumor antigen binding cell surface receptor targeting the at least one tumor antigen identified to be expressed or overexpressed in the solid tumor of said subject.

In another aspect the present invention relates to a method for treating a solid tumor in a subject comprising administration of a Salmonella typhi Ty21 a strain comprising a DNA molecule comprising at least one eukaryotic expression cassette encoding at least one polypeptide comprising five or more neoantigens to the subject, wherein the subject has been or is treated with at least one engineered T cell, NKT cell or NK cell comprising at least one tumor antigen binding cell surface receptor. More specifically, the method comprises administering at least one engineered T cell, NKT cell or NK cell comprising at least one tumor antigen binding cell surface receptor followed by administering a Salmonella typhi Ty21 a strain comprising a DNA molecule comprising at least one eukaryotic expression cassette encoding at least one polypeptide comprising five or more neoantigens to the subject. The at least one engineered T cell, NKT cell or NK cell comprising at least one tumor antigen binding cell surface receptor is administered to the subject by adoptive cell transfer. The method may further comprise identifying at least five or more neoantigens are tumor specific antigens in the solid tumor of said subject and generating the Salmonella typhi Ty21a strain comprising a DNA molecule comprising at least one eukaryotic expression cassette encoding at least one polypeptide comprising five or more neoantigens. The method may further comprise identifying at least one tumor antigen identified to be expressed or overexpressed in the solid tumor of said subject and administering at least one engineered T cell, NKT cell or NK cell comprising at least one tumor antigen binding cell surface receptor targeting the at least one tumor antigen identified to be expressed or overexpressed in the solid tumor of said subject.

According to the invention, the attenuated Salmonella strain functions as the bacterial carrier of the DNA molecule comprising at least one eukaryotic expression cassette encoding at least one polypeptide comprising five or more neoantigens for the delivery of said DNA molecule into a target cell. Preferably the DNA molecule is part of a plasmid comprising the DNA molecule comprising at least one eukaryotic expression cassette encoding at least one polypeptide comprising five or more neoantigens. Such a bacterial carrier or delivery vector comprising the DNA molecule comprising at least one eukaryotic expression cassette encoding at least one polypeptide comprising five or more neoantigens may also be referred to as DNA vaccine.

In the context of the present invention, the term “vaccine” refers to an agent which is able to induce an immune response in a subject upon administration. A vaccine can preferably prevent, ameliorate or treat a disease. In the context of the present invention the vaccine is preferably an oral vaccine. The Salmonella typhi Ty21a strain comprising a DNA molecule comprising at least one eukaryotic expression cassette encoding at least one polypeptide comprising five or more neoantigens according to the invention may be abbreviated to “Salmonella typhi Ty21a strain encoding at least one polypeptide comprising five or more neoantigens” or “neoantigen cancer vaccine”.

### Neoantigen Cancer Vaccine

The live attenuated Salmonella strain, more particularly the Salmonella typhi Ty21a strain, according to the present invention stably carries a recombinant DNA molecule comprising at least one eukaryotic expression cassette encoding at least one polypeptide comprising five or more neoantigens. It is used as a carrier for oral delivery of this recombinant DNA molecule. The term “an attenuated strain of Salmonella typhi Ty21a” as used herein refers to an attenuated strain of Salmonella, more specifically of Salmonella typhi, wherein the attenuated strain is Ty21a and is used synonymously with “Salmonella typhi Ty21a” herein.

Genetic immunization might be advantageous over conventional vaccination. The target DNA can be detected for a considerable period of time thus acting as a depot for the antigen. Sequence motifs in some plasmids, like CpG islands, are immunostimulatory and can function as adjuvants furthered by the immunostimulation due to LPS and other bacterial components.

Live attenuated Salmonella vectors produce their own immunomodulatory factors such as lipopolysaccharides (LPS) in situ which may constitute an advantage over other forms of administration such as microencapsulation. Moreover, the mucosal vaccine according to the present invention has an intra-lymphatic mode of action, which proved to be beneficial. After ingestion of the attenuated vaccine according to the present invention, macrophages and other cells in Peyer's patches of the gut are invaded by the modified bacteria. The bacteria are taken up by these phagocytic cells. Due to their attenuating mutations, bacteria of the S. typhi Ty21 strain are not able to persist in these phagocytic cells and die. The recombinant DNA molecules are released and subsequently transferred into the cytosol of the phagocytic immune cells, either via a specific transport system or by endosomal leakage. Finally, the recombinant DNA molecules enter the nucleus, where they are transcribed, leading to massive expression of the polypeptide(s) comprising five or more neoantigens in the cytosol of the phagocytic cells. The infected cells undergo apoptosis, loaded with the polypeptide(s) comprising five or more neoantigens, and are taken up and processed by the gut's immune system. The danger signals of the bacterial infection serve as a strong adjuvant in this process, leading to a strong target antigen specific CD8+ T-cell and antibody response at the level of both systemic and mucosal compartments. The immune response peaks around ten days after vaccination. The lack of anti-carrier response allows boosting with the same vaccine several times.

In the context of the present invention, the term “attenuated” refers to a bacterial strain of reduced virulence due to an attenuating mutation compared to the parental bacterial strain, not harboring the attenuating mutation. Attenuated bacterial strains have preferably lost their virulence but retain their ability to induce protective immunity. Attenuation can be accomplished by deletion of various genes, including virulence, regulatory, and metabolic genes. Attenuated bacteria may be found naturally or they may be produced artificially in the laboratory, for example by adaptation to a new medium or cell culture or they may be produced by recombinant DNA technology. Administration of about 10¹¹ CFU of the attenuated strain of Salmonella according to the present invention preferably causes Salmonellosis in less than 5%, more preferably less than 1%, most preferably less than 1%. of subjects. The strain Ty21a according to the invention is an attenuated strain of Salmonella typhi.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. In one embodiment the term “comprising” as used throughout the application and in particular within the claims may be replaced by the term “consisting of”. The term “a” as used herein may include the plural and includes, but is not limited to “one”.

The term “neoantigens” as used herein relates to peptides that are generated from somatically mutated genes expressed only in cancer cells, but not in normal tissue of the same patient. Genes and chromosomes can mutate in either somatic or germinal tissue. Opposite to germline mutations, somatic mutations are not transmitted to progeny. Thus, the somatic mutations in the gene have been acquired in the cancer cells and during cancer development. Typically the mutation is a tumor-specific point mutation generating a neoepitope also referred to as a mutational epitope or point-mutated peptide. They are highly immunogenic because they are not present in normal tissues and hence bypass central thymic tolerance. Neoantigens comprise, preferably consist, of the neoepitope presented as peptide by MHC I or MHC II. The mutation may also be a frameshift mutation resulting in a frameshift peptide (FSP) antigen. FSP neoantigens, although caused by insertion or deletion of single nucleotides, encompass long antigenic amino acid stretches, which can contain multiple immunologically relevant neoepitopes. In specific embodiments, the term “neoantigen” further includes T cell epitopes associated with peptide processing (TEIPP). TEIPPs are derived from ubiquitously expressed non-mutated “self” proteins that are not loaded into MHC I in healthy cells. In immune-escaping cancers antigen-processing components, like the transporter associated with antigen processing (TAP) are often downregulated. Thus, only in cells with defects in the antigen-processing machinery, such as in the absence of TAP due to mutations or epigenetic silencing, TEIPPs may be presented on the surface of cancer cells (Marjit et al., Journal of Experimental Medicine, 2018, 215(9): 2325).

During cancer progression, mutations accumulating in the cancer genome can affect protein-coding genes and result in altered protein sequences. Mutated proteins are proteolytically cleaved into short peptides and presented on the tumor cell surface by MHC (human leucocyte antigen (HLA) in humans). These somatically mutated genes, i.e., neoantigens, which are presented in the malignant cells but not in the normal cells can be recognized as foreign by tumor-infiltrating lymphocytes (TILs). Thus, the term neoantigen refers to a peptide comprising, preferably consisting of the peptide containing the somatic mutation that is presented by MHC I or II. Neoantigens presented by MHC I may also be referred to as CD8 T cell antigens. Neoantigens presented by MHC II may also be referred to as CD4 T cell antigens (or T helper antigens). As neoantigens can be recognized as foreign by TILs they are capable of eliciting potent tumor specific immune responses. Neoantigens released after tumor cell death initiate a number of processes that ultimately lead to T cells that recognize cancer cells through the interaction of distinct T-cell receptors (TCR) with specific neoantigen-MHC complexes.

The term “at least one polypeptide comprising five or more neoantigens” as used herein refers to one polypeptide or more than one polypeptide comprising together 5 or more neoantigens. Whether the five or more neoantigens are part of the same or different polypeptides is not relevant. The five or more neoantigens may therefore be expressed as one polypeptide or as more than one polypeptide. Preferably the neoantigens comprised within the at least one or more polypeptide(s) are 10 or more, 20 or more, 30 or more, 50 or more, or more than 50 neoantigens. In the context of the Salmonella typhi Ty21a strain as used herein, the insert encoding the at least one polypeptide may comprise up to 300 neoantigens, preferably up to 200 neoantigens. Antigens presented as peptides on MHC class I or II (in humans HLA) are typically from 11 to 30 amino acids long for MHC II (CD4 antigens) and from 8 to 10 amino acids for MHC I (CD8 antigens). Thus, preferred ranges for neoantigens to be contained within the at least one polypeptide are 5 to 300, 10 to 300, 20 to 300, 30 to 300, 50 to 300, or more than 50 to 300 neoantigens. More preferred ranges for neoantigens to be contained within the at least one polypeptide are 5 to 200, 10 to 200, 20 to 200, 30 to 200, 50 to 200, or more than 50 to 200 neoantigens. Each polypeptide comprising fused neoantigens is proteolytically cleaved into the neoantigens inside antigen presenting cells and presented via HLA to elicit a T-cell response.

According to the invention, the five or more neoantigens may comprise CD8 T cell antigens and/or CD4 T cell antigens. Preferably, the five or more neoantigens comprise CD8 T cell antigens and CD4 T cells antigens.

It is hypothesized that vaccination with neoantigens can both expand pre-existing neoantigen-specific T-cell populations and induce a broader repertoire of new T-cell specificities in cancer patients.

A neoantigen is typically a peptide having 8 to 30 amino acids, preferably 8 to 20, more preferably 8 to 12 amino acids.

For a neoantigen cancer vaccine it is beneficial if the vaccine targets multiple neoantigens, thus reducing the risk of immune-evasion due to loss of expression of subsets of neoantigens. It is also encompassed by the invention that the patient is treated sequentially with another Salmonella typhi Ty21a strain comprising a DNA molecule comprising at least one eukaryotic expression cassette encoding at least one polypeptide comprising five or more neoantigens, comprising targeting new neoantigens or a new subset of neoantigens selected during tumor progression.

Advantage of the attenuated strain of Salmonella typhi Ty21a, also referred to as “Salmonella typhi Ty21a”, as carrier for the at least one polypeptide comprising five or more neoantigens are the established quality control assay, the individual differences of the plasmid only in the insert encoding the one or more neoantigens, no need for expansion and no requirements with regard to sterility testing due to oral administration. Furthermore, expression plasmids suitable for transformation as well as the Salmonella typhi Ty21a strain as carrier allow a high number (up to 300) of epitopes (neoantigens). The neoantigens may be inserted into the plasmid as a string of beads (expressed as one or more polypeptides), optionally separated by a linker. The linker may be, without being limited thereto, a GS linker, a 2A cleavage site, or an IRES sequence. Due to the fast generation and only limited need for quality control, the time for generating the Salmonella typhi Ty21a strain comprising a DNA molecule comprising at least one eukaryotic expression cassette encoding at least one polypeptide comprising five or more neoantigens is short and can for example be achieved within 15 days, preferably within 14 days or less after identification of the neoantigens. Overnight fermentation is sufficient and no upscaling is required due to high yield of bacteria with a net yield in the range of 10¹¹ colony forming units (CFU) in a 1L culture. This allows for the short manufacturing time, as well as the low manufacturing costs. Furthermore, each batch is sufficient for years of treatment and the drug product was shown to be stable for at least three years. Thus, no batch variation will occur, since one batch lasts for the entire treatment of the subject having the solid tumor.

A method for generating a Salmonella typhi Ty21a strain comprising a DNA molecule comprising at least one eukaryotic expression cassette encoding at least one polypeptide comprising five or more neoantigens for an individual subject with a solid tumor comprises (a) providing a tumor cell sample and a control sample from said subject; (b) identifying five or more neoantigen present in the tumor cell sample that are not present in the control sample; (c) selecting five or more neoantigens (d) synthesizing a cDNA encoding the at least one polypeptide comprising five or more neoantigens; (e) cloning the cDNA into the at least one eukaryotic expression cassette (f) transforming a Salmonella typhi Ty21a recipient strain with the DNA molecule comprising at least one eukaryotic expression cassette encoding at least one polypeptide comprising five or more neoantigens; (f) fermenting the strain obtained in step (f) and diluting to target concentration based on CFU; and (g) analyzing said transformed Salmonella typhi Ty21a strain comprising sequencing the cDNA encoding the at least one polypeptide comprising five or more neoantigens. The control sample may be any sample of normal tissue or blood from the subject to be treated. Preferably the control sample is a blood sample. The blood sample may further be used for HLA typing of the patient. The tumor cell sample may be a tumor biopsy.

Methods for detecting (all) coding mutations within tumors and reliably predicting or determining those mutated peptides with high-affinity binding of autologous human leukocyte antigen (HLA) molecules are known in the art. For example whole-exome sequencing (WES) of matched tumor and normal cell DNA from individual patients can be performed. Identified somatic mutations are then orthogonally validated and assessed for expression of mutated alleles by RNA sequencing of the tumor. Peptides are then selected that are predicted to likely bind to autologous HLA-A or HLA-B proteins of the patient. This may be confirmed, e.g., by ex vivo interferon γ enzyme-linked immunospot (ELISPOT). Alternatively, HLA-peptide ligands can be isolated from cell media and identification can be conducted by LC-MS/MS analysis.

A polypeptide may comprise several neoantigens fused to each other, preferably 5 or more, 10 or more, 20 or more, 30 or more, or 50 or more neoantigens. In a typical plasmid used to transfect the Salmonella typhi Ty21a strain, such as pVAX1™ expression plasmid (Invitrogen, San Diego, Calif.) or pVAX10 derived thereof, up to about 300 neoantigens may be expressed. The polypeptide may therefore comprise about 5 to 300, 10 to 300, 20 to 300, 30 to 300 or 50 to 300 neoantigens, preferably 10 to 200, 20 to 200, 30 to 300, or 50 to 200 neoantigens. The polypeptide is cleaved intracellularly into peptide and presented on MHC I or MHC II molecules, depending on the type of neoantigen. The individual neoantigens may be separated by a linker, such as a GS linker, specifically designed linkers or a 2A cleavage site. The DNA molecule encoding the neoantigen(s) may also be separated by an IRES sequence, resulting in separate polypeptides.

The Salmonella typhi Ty21a strain comprising a DNA molecule comprising at least one eukaryotic expression cassette encoding at least one polypeptide comprising five or more neoantigens may further comprising a DNA molecule comprising at least one eukaryotic expression cassette encoding at least one polypeptide comprising at least one tumor antigen that is not a neoantigen and/or tumor stromaantigen, wherein said at least one tumor antigen that is not a neoantigen is expressed in the solid tumor of the patient to be treated. The at least one polypeptide comprising at least one tumor antigen that is not a neoantigen and/or tumor stroma antigen (a) may be encoded by the same DNA molecule comprising the at least one eukaryotic expression cassette encoding the at least one polypeptide comprising five or more neoantigens or by a further separate DNA molecule, (b) may be encoded by the at least one eukaryotic expression cassette encoding the at least one polypeptide comprising five or more neoantigens or by a further separate expression cassette; or (c) may be the at least one polypeptide comprising five or more neoantigens or a further separate polypeptide. Thus, the Salmonella typhi Ty21a strain may be transformed with two DNA molecules, the first encoding the five or more neoantigens and the second encoding the at least one tumor antigen that is not a neoantigen and/or tumor stroma antigen. Alternatively, the Salmonella typhi Ty21a strain may be transformed with one DNA molecules, comprising at least one eukaryotic expression cassette encoding at least one polypeptide comprising five or more neoantigens and at least one further eukaryotic expression cassette encoding at least one tumor antigen that is not a neoantigen and/or tumor stroma antigen. Alternatively, the Salmonella typhi Ty21a strain may also be transformed with one DNA molecules, comprising at least one eukaryotic expression cassette encoding at least one polypeptide comprising five or more neoantigens and further comprising at least one tumor antigen that is not a neoantigen and/or tumor stroma antigen. Examples for tumor antigens in this context are, without being limited thereto, WT1, MSLN, CEA, HER2, EGFR, FBP, GD2, GD3, MAGE-A1, PSCA, PSMA, MUC1, GPC3 and CMV pp65. The tumor antigen may be a tumor specific antigen or a tumor associated antigen. The term “tumor specific antigen” as used herein relates to an antigen expressed in the tumor, but not in normal tissue. The term “tumor associated antigen” as used herein relates to an antigen overexpressed in the tumor compared to normal tissue. The term “tumor stroma antigen” as used herein refers to antigens expressed in the tumor stroma including, without being limited thereto, VEGFR-2 and FAP. The Salmonella typhi Ty21a strain comprising a DNA molecule comprising at least one eukaryotic expression cassette encoding at least one polypeptide comprising five or more neoantigens may also comprising a DNA molecule comprising at least one eukaryotic expression cassette encoding at least one polypeptide comprising a checkpoint inhibitor antigen, wherein said at least one checkpoint inhibitor antigen or its ligand is overexpressed in the solid tumor of the patient to be treated. With regard to the expression of the checkpoint inhibitor antigen in the Salmonella typhi Ty21a strain comprising a DNA molecule comprising at least one eukaryotic expression cassette encoding at least one polypeptide comprising five or more neoantigens, the same applies as for the at least one tumor antigen that is not a neoantigen and/or the tumor stroma antigen. An example for a checkpoint inhibitor antigen is PD-1 or PD-L1. The DNA molecule used in this context is preferably an expression plasmid.

A DNA molecule comprising at least one eukaryotic expression cassette encoding at least one polypeptide comprising five or more neoantigens may also be referred to as a recombinant DNA molecule, i.e. an engineered DNA construct, preferably composed of DNA pieces of different origin. The DNA molecule can be a linear nucleic acid, or preferably, a circular DNA plasmid generated by introducing an open reading frame encoding at least one polypeptide comprising five or more neoantigens into a eukaryotic expression cassette of a plasmid. A plasmid comprising a eukaryotic expression cassette may also be referred to as eukaryotic expression plasmid.

In the context of the present invention, the term “expression cassette” refers to a nucleic acid unit comprising at least one open reading frame (ORF) under the control of regulatory sequences controlling its expression. Expression cassettes can preferably mediate transcription of the included open reading frame encoding at least one polypeptide comprising five or more neoantigens, in a eukaryotic target cell. Eukaryotic expression cassettes typically comprise a promoter, at least one open reading frame and a transcription termination signal, which allow expression in a eukaryotic target cell.

In particular embodiments, a single dose of the Salmonella typhi Ty21a strain comprises from about 10⁶ to about 10¹⁰, more particularly from about 10⁶ to about 10⁹, more particularly from about 10⁷ to about 10⁹, more particularly from about 10⁶ to about 10⁸, most particularly from about 10⁶ to about 10⁷ colony forming units (CFU).

More particularly, a single dose of the Salmonella typhi Ty21a strain comprises from about 1×10⁶ to about 1×10¹⁰, more particularly from about 1×10⁶ to about 1×10⁹, more particularly from about 1×10⁷ to about 1×10⁹, more particularly from about 1×10⁶ to about 1×10⁸, most particularly from about 1×10⁶ to about 1×10⁷ colony forming units (CFU).

Furthermore, the Salmonella typhi Ty21a strain according to the invention is preferably administered two to four times in the first week, preferably 4 times in the first week, followed by single dose boosting administration every 2 to 4 weeks, particularly on day 1 and 7, preferably on day 1, 3, 5 and 7 followed by single dose boosting administrations every 2 to 4 weeks.

In this context, the term “about” or “approximately” means within a factor of 3, alternatively within a factor of 2, including within a factor of 1.5 of a given value or range.

In particular embodiments, the treatment comprises a single or multiple administrations of the Salmonella typhi Ty21a strain encoding at least one polypeptide comprising five or more neoantigens or the pharmaceutical composition according to the present invention. The single dose of the administrations may be the same or different, preferably the same and preferably within the ranges as disclosed herein. In particular, the treatment comprises two to four prime vaccinations in the first week of treatment followed by single dose boosting administrations every two to four weeks of the Salmonella typhi Ty21a strain encoding at least one polypeptide comprising five or more neoantigens or the pharmaceutical composition according to the present invention.

The Salmonella typhi Ty21a strain encoding at least one polypeptide comprising five or more neoantigens is for use in the treatment of a solid tumor in a subject, wherein the subject has been or is treated with at least one engineered T cell, NKT cell or NK cell comprising at least one tumor antigen binding cell surface receptor.

The solid tumor to be treated in accordance to the invention may be any solid tumor, particularly a solid tumor selected from colorectal cancer, pancreatic cancer, lung cancer, ovarian cancer, mesothelioma, glioblastoma, gastric cancer, hepatocellular cancer, renal cell cancer, prostate cancer, cervical cancer, breast cancer and melanoma.

### Engineered T Cells, NKT Cells or NK Cells

The Salmonella typhi Ty21a strain comprising a DNA molecule comprising at least one eukaryotic expression cassette encoding at least one polypeptide comprising five or more neoantigens is administered to a subject having a solid tumor for use in treating the solid tumor. In one embodiment, the subject has been or is further treated with at least one engineered T cell, NKT cell or NK cell comprising at least one tumor antigen binding cell surface receptor. Preferably at least one engineered T cell, NKT cell or NK cell comprising at least one tumor antigen binding cell surface receptor is provided to the subject having a solid tumor by adoptive cell transfer (ACT), i.e., by injection of the at least one engineered T cell, NKT cell or NK cell intravenously. The term “adoptive cell transfer” as used herein refers to the transfer of cells into a patient or a subject. In the context of the at least one engineered T cell, NKT cell or NK cell it is used synonymously with “administration” or “to be administered”. The term “at least one engineered T cell, NKT cell or NK cell comprising at least one tumor antigen binding cell surface receptor” as used herein means “at least one engineered T cell comprising at least one tumor antigen binding cell surface receptor, at least one engineered NKT cell comprising at least one tumor antigen binding cell surface receptor or at least one engineered NK cell comprising at least one tumor antigen binding cell surface receptor” and may be abbreviated to “at least one engineered T cell, NKT cell or NK cell” or “at least one engineered T cell, at least one engineered NKT cell or at least one engineered NK cell”. More particularly, the at least one engineered T cell comprising at least one tumor antigen binding cell surface receptor may be an engineered conventional αβT cell or an engineered γδ T cell, preferably the at least one engineered T cell comprising at least one tumor antigen binding cell surface receptor is an engineered conventional αμ T cell.

The at least one engineered T cell, NKT cell or NK cell comprising at least one tumor antigen binding cell surface receptor is usually custom made. This requires identifying a tumor antigen expressed in a solid tumor of the subject to be treated. Identifying a tumor antigen expressed in a solid tumor of the subject to be treated comprises (a) providing a tumor cell sample and a control sample from said subject; and (b) identifying a tumor antigen expressed in the tumor cell sample that is not present in the control sample. The tumor cell sample may be a tumor biopsy. Furthermore, a tumor cell sample is to be understood to contain solid tumor tissue as well as tumor stroma tissue. Identifying the tumor antigen expressed in the solid tumor of the solid tumor of the subject to be treated for generating the at least one engineered T cell, NKT cell or NK cell and identifying the five or more neoantigens expressed in a solid tumor of the subject to be treated for generating the Salmonella typhi Ty21a strain encoding at least one polypeptide comprising five or more neoantigens can be performed at the same time or at separate time points and/or can be performed in the same tumor cell sample or in a different tumor cell samples. Preferably, identifying the five or more neoantigens and the tumor antigen and/or tumor stroma antigen expressed in the solid tumor or the stroma of the solid tumor of the subject to be treated comprises (a) providing a tumor cell sample and a control sample from said subject; and (b) identifying five or more neoantigen and a tumor antigen and/or tumor stroma antigen present in the tumor cell sample that are not present in the control sample. The tumor antigen targeted by the at least one tumor antigen binding cell surface receptor may be the same or different to at least one of the neoantigens targeted by the Salmonella typhi Ty21a strain comprising a DNA molecule comprising at least one eukaryotic expression cassette encoding at least one polypeptide comprising five or more neoantigens.

The at least one engineered T cell, NKT cell or NK cell comprising at least one tumor antigen binding cell surface receptor may be an autologous or an allogenic T cell, NKT cell or NK cell. The term “autologous means that the T cell, NKT cell or NK cell is obtained from the patient, genetically engineered by introducing a nucleic acid molecule encoding at least one tumor antigen binding cell surface receptor, expanded in vitro and transferred back to the subject to be treated. Preferably the engineered T-cell is an autologous T-cell, i.e., originating from the subject to be treated. The T cell, particularly the engineered conventional αβT cell, may also be allogenic to the subject to be treated, i.e., from another healthy donor. Engineered NKT cell as well as engineered NK cell may be autologous or allogenic NKT cell or NK cell, respectively. Adoptive cell transfer of T cells (particularly of conventional αβT cells) has some risk of graft versus host disease (GvHD), while this may be less the case with adoptive cell transfer of NKT cells, NK cells or γδ T cells. Allogenic engineered T cells, NKT cells, or NK cells comprising at least one tumor antigen binding cell surface receptor may be pre-made or an off-the-shelf product. In addition, for NK cells the use of allogenic cells has the advantage that NK cells are less inhibited by KIR signaling triggered by self-MHC molecules.

The term “engineered” as used herein means “modified” to express the at least one tumor antigen binding cell surface receptor on the cell surface. The gene or mRNA that encodes the at least one tumor antigen binding cell surface receptor on the cell surface is introduced into the T cell, NKT cell or NK cell, preferably by transfection or transduction. The T cell, NKT cell or NK cell may be transfected or transduced with RNA or DNA encoding the at least one tumor antigen binding cell surface receptor. The engineered T cells, NKT cells or NK cells, are then multiplied or expanded ex vivo prior to be infused back into the subject. Suitable vectors for gene delivery are known in the art and include for example viral vectors, such as retroviral and lentiviral vectors, or transposons such as Piggy-Bac (PB) and Sleeping Beauty (SB). RNA transiently-engineered T cell, NKT cell or NK cell, preferably CAR-T cells, CAR-NKT cell or CAR-NK cells are also envisaged by the present invention.

In certain embodiments, the at least one engineered T cell, NKT cell or NK cell comprises at least one tumor antigen binding cell surface receptor on its cell surface, wherein the tumor antigen is selected from carcinoembryonic antigen (CEA), epithelial growth factor receptor (EGFR), folate binding protein (FBP), GD2, GD3, human epidermal growth factor receptor 2 (HER2, erb-B2), melanoma antigen A1 (MAGE-A1), mesothelin (MSLN), prostate stem cell antigen (PSCA), prostate-specific membrane antigen (PSMA), mucin-1 (MUC1), glypican-3 (GPC3), Wilm's tumor protein (WT1), epithelial cell adhesion molecule (EpCAM), B-cell maturation antigen (BCMA) and tyrosine-protein kinase transmembrane receptor (ROR1). Wherein the tumor antigen may for example be expressed, without being limited thereto, by the solid tumors as listed in Table 1:

| Tumor antigen | Solid tumor |
|---|---|
| CEA | Colorectal cancer, breast cancer, hepatocellular cancer |
| EGFR | Glioma; lung cancer, particularly non-small cell lung |
| | cancer |
| FBP | Ovarian cancer |
| GD2 | Neuroblastoma, glioblastoma |
| GD3 | Glioblastoma, melanoma |
| HER2 | Carcinomas such as glioblastoma, glioma, sarcoma, |
| | head and neck squamous cell carcinoma, breast cancer, |
| | ovarian cancer, gastric cancer, lung cancer, pancreatic |
| | cancer |
| MAGE-Al | Lung cancer, melanoma, head and neck cancer |
| MSLN | Metastatic cancer, mesothelioma, pancreatic cancer, |
| | breast cancer, lung cancer |
| PSCA | Prostate cancer |
| PSMA | Prostate cancer |
| MUC1 | Carcinomas such as glioblastoma, glioma, breast |
| | cancer, gastric cancer, lung cancer, pancreatic cancer, |
| | colorectal cancer, hepatocellular cancer |
| GPC3 | Lung cancer, particularly non-small cell lung cancer, |
| | hepatocellular cancer |
| WT1 | Glioma, ovarian cancer, lung cancer |
| EpCAM | Colorectal cancer, renal cancer, prostate cancer |
| BCMA | Breast cancer |
| ROR1 | Ovarian cancer |

The term “engineered T cell comprising at least one tumor antigen binding cell surface receptor” refers to a T cell carrying a recombinant T cell receptor binding to a tumor antigen. Particularly, this means a T cell carrying a recombinant surface receptor comprising at least one tumor antigen binding domain, an activation domain and a co-stimulatory domain. Preferably this means a T cell carrying a chimeric antigen receptor (CAR), a so called “CAR-T cell”. As used herein an engineered T cell comprising at least one tumor antigen binding cell surface receptor or a CAR-T cell may be understood to refer specifically to an engineered conventional αβT cell or CAR-αβT cell, respectively. In addition or alternatively an engineered T cell comprising at least one tumor antigen binding cell surface receptor or a CAR-T cell may be an engineered conventional αβT cell or CAR-αβT cell or an engineered γδ T cell or CAR-0 T cell. In the context of the present invention engineered T cells or CAR-T cells may comprise small amounts of other subsets of T cells, such as NKT cells or CAR-NKT cells. Typically the engineered T cells or CAR-T cells according to the present invention contain NKT cells or CAR-NKT cells, respectively, at less than 5%, less than 2%, less than 1% or less than 0.5%. The term “at least one engineered T cell comprising at least one tumor antigen binding cell surface receptor” includes a population of engineered T cell, preferably a substantially pure population of engineered T cells, more preferably a mixture of cells comprising more than 50%, more than 60%, more than 70%, more than 80%, more than 90% or more than 95% T cells. The engineered T cell is preferably generated from peripheral blood (PB), bone marrow (BM), cord blood (CB), placenta or induced pluripotent stem cells (iPSC) from a different donor or the subject to be treated.

The term “engineered NKT cell comprising at least one tumor antigen binding cell surface receptor” refers to a NKT cell carrying a recombinant surface receptor binding a tumor antigen, particularly, a NKT cell carrying a recombinant surface receptor comprising at least one tumor antigen binding domain, an activation domain and a co-stimulatory domain. Preferably this means a NKT cell carrying a chimeric antigen receptor (CAR), a so called “CAR-NKT cell”. NKT cells have a T cell receptor that recognizes glycolipids and lipids presented via the non-classical MHC protein CD1d. The term “NKT cell” as used herein refers to CD1d-restricted T cells. NKT cells are a subset of T cells that coexpress an αβT cell receptor, but also express a variety of molecular markers that are typically associated with NK cells, such as NK1.1. NKT cells are subdivided into NKT cells with an invariant T cell receptor (invariant or typel NKT cells) and diverse NKT cells (type 2 NKT cells). CAR-NKT cells have the advantage that the CAR activity may synergize with the intrinsic antitumor activity of NKT cells. The term “at least one engineered NKT cell comprising at least one tumor antigen binding cell surface receptor” includes a population of engineered NKT cell, preferably a substantially pure population of engineered NKT cells, more preferably a mixture of cells comprising more than 50%, more than 60%, more than 70%, more than 80%, more than 90% or more than 95% NKT cells. The engineered NKT cell is preferably generated from peripheral blood (PB), bone marrow (BM), cord blood (CB), placenta or induced pluripotent stem cells (iPSC) from a different donor or the subject to be treated.

The term “engineered NK cell comprising at least one tumor antigen binding cell surface receptor” refers to a NK cell carrying a recombinant surface receptor binding a tumor antigen, particularly, a NK cell carrying a recombinant surface receptor comprising at least one tumor antigen binding domain, an activation domain and a co-stimulatory domain. Preferably this means a NK cell carrying a chimeric antigen receptor (CAR), a so called “CAR-NK cell”. NK cells are lymphocytes comprising a number of activatory and inhibitory germline-encoded receptors. Those receptors include the NKG2D receptor recognizing the stress ligands MIC-A and MIC-B on tumor cells and the natural cytotoxicity receptors NKp30, 46 and p44. On the other hand the killer cell immunoglobulin-like receptor (KIR) family binds to class I MHC molecules and inhibit NK cell activation. There are two major mechanisms to evoke NK effector functions: (i) missing self: absence of inhibitory ligands, such as a result of downregulated MHC presentation, and (ii) induced self: pro-activatory stimuli outweigh their inhibitory counterparts, such as by upregulation of stress ligands or cells coated by antibodies. The term “at least one engineered NK cell comprising at least one tumor antigen binding cell surface receptor” includes a population of engineered NK cell, preferably a substantially pure population of engineered NK cells, more preferably a mixture of cells comprising more than 50%, more than 60%, more than 70%, more than 80%, more than 90% or more than 95% NK cells. The engineered NK cell is preferably generated from peripheral blood (PB), bone marrow (BM), cord blood (CB), placenta or induced pluripotent stem cells (iPSC) from a different donor or the subject to be treated or from an NK cell line (e.g., NK-92 cells). CAR-NK cells have the advantage that the CAR activity may synergize with the intrinsic antitumor activity of NK cells.

In preferred embodiments, the at least one engineered T cell, NKT cell or NK cell comprising at least one tumor antigen binding cell surface receptor is a CAR-T cell, a CAR-NKT cell or a CAR-NK cell.

The term “tumor antigen binding cell surface receptor” refers to a recombinant surface receptor comprising at least one tumor antigen binding domain, preferably to a chimeric antigen receptor (CAR). A CAR construct typically consists of three components, an extracellular antigen-recognition domain, a transmembrane domain and an intracellular signaling domain. The extracellular domain comprises the antigen-recognition site and is usually composed of a single-chain variable fragment (scFv). In certain embodiments the extracellular domain may also comprise two or more scFv with the same or preferably different antigen specificity. It is typically linked to the transmembrane domain via a hinge region, which imparts flexibility for adequate orientation and binding to the antigen. The intracellular signaling domain comprises a stimulatory domain, such as an intracellular domain of an activatory receptor (e.g., CD3ζ or FcRγ) and preferably at least one co-stimulatory domain, such as an intracellular domain of as co-stimulatory receptor (e.g. CD28 or 4-1BB). CARs are artificial cell surface receptors, i.e., transmembrane proteins, comprising an extracellular ligand recognition domain, an intracellular signaling domain which activates the respective cells (e.g., CD3ζ or FcRγ) and preferably at least one intracellular domain of a co-stimulatory receptor (e.g., CD28 or 4-1BB). They are called chimeric because they are fused of parts from different sources. The extracellular ligand recognition domain is preferably based on the specificity of a monoclonal antibody and is typically a single-chain variable fragment (scFv).

CARs encode for transmembrane chimeric molecules with dual function: (a) immune recognition of tumor antigens expressed on the surface of tumor cells, and (b) active promotion and propagation of signaling events controlling the activation of the activatory and/or lytic machinery. This system has several advantages: (1) to provide “reprogrammed T cells”, “reprogrammed NKT cell” or “reprogrammed NK cells” of an ex-novo activation mechanism, (2) to break tolerance acquired by tumor cells, and (3) to bypass restrictions of the HLA-mediated antigen recognition, over-stepping one of the barriers to a more widespread application of cellular immunotherapy.

For T cells CARs may be, e.g., chimeric fusion proteins comprising a scFv as extracellular ligand recognition domain and an intracellular signaling domain comprising a CD3ζ-chain signaling domain or a FcRγ-chain signaling domain. This provides T-lymphocytes with antibody-type specificity and activates all functions of an effector cell, including the production of cytokines, such as IL-2, and the lysis of target cells. CARs may further comprise an intracellular CD28 costimulatory domain and/or an additional transducer domain, such as from CD27, 4-1BB, CD40L, PD-1 or OX40. Typically the CARs are predesigned and/or provided as DNA or RNA molecules encoding the CARs. Thus, although the tumor antigen targeted by the CAR-T cell may theoretically also be a neoantigen, it is typically a tumor antigen that is expressed or overexpressed in certain solid tumors and identified to be expressed in the solid tumor of the subject to be treated.

According to the invention engineered T-cell comprising at least one tumor antigen binding cell surface receptor also include “armored CAR-T cells”. Armored CAR-T cells have been further optimized to inducibly or constitutively secrete active cytokines, such as interleukins or express ligands that further armor the CAR T-cells to improve efficacy and persistence. The choice of the “armor” agent is based on the knowledge of the tumor microenvironment and the roles of other elements of the innate and adaptive immune system. Examples are interleukine-2 (IL-2), interleukine-12 (IL-12), interleukine-15 (IL-15), CD40L and 4-1BBL. These agents have been shown to further enhance CAR T-cells efficacy and persistence in tumor microenvironment via different mechanisms. These are typically expressed as an independent gene in the same CAR vector.

The two recently approved CAR-T cells KYMRIAH (tisagenlecleucel) and YESCARTA (axicabtagene ciloleucel) both contain an anti-CD19 murine scFv, but they signal through different costimulatory domains fused in tandem with the CD3 ζ-chain: 4-1BB for KYMRIAH, and CD28 for YESCARTA.

For NKT cells CARs may be, e.g., chimeric fusion proteins comprising a scFv as extracellular ligand recognition domain and an intracellular signaling domain comprising a CD3 ζ-chain signaling domain or a FcRγ-chain signaling domain. This provides NKT cells with antibody-type specificity and activates all functions of an effector cell. For NKT cells this includes the production of IFNγ and granulocyte-macrophage colony stimulating factor (GM-CSF) and the lysis of target cells based on perforin and Fas ligand mediated killing. CARs may further comprise an intracellular CD28 costimulatory domain and/or an additional transducer domain, such as from CD27, CD40L, PD-1, 4-1 BB or OX40. Typically the CARs are predesigned and/or provided as DNA or RNA molecules encoding the CARs. Thus, although the tumor antigen targeted by the CAR-NKT cell may theoretically also be a neoantigen, it is typically a tumor antigen that is expressed or overexpressed in certain solid tumors and identified to be expressed in the solid tumor of the subject to be treated.

For NK cells CARs may be, e.g., chimeric fusion proteins comprising a scFv as extracellular ligand recognition domain and an intracellular signaling domain comprising a CD3 ζ-chain signaling domain or a FcRγ-chain signaling domain. This provides NK cells with antibody-type specificity and activates all functions of an effector cell. For NK cells this includes the production of IFNγ and granulocyte-macrophage colony stimulating factor (GM-CSF) and the lysis of target cells. CARs may further comprise an intracellular CD28 costimulatory domain and/or an additional transducer domain, such as from CD27, CD40L, PD-1, 4-1BB, 4-1BB, OX40 or 2B4 (CD244) or a DNAX-activation protein 12 (DAP12) domain. A key cytokine in the protective tumor microenvironment is transforming growth factor beta (TGF-β), which inhibits NK cells. Thus, fusing the extracellular domain of TGF-β receptor to the intracellular domain of the NKG2D receptor may further improve the effect of CAR-NK cells. Typically the CARs are predesigned and/or provided as DNA or RNA molecules encoding the CARs. Thus, although the tumor antigen targeted by the CAR-NK cell may theoretically also be a neoantigen, it is typically a tumor antigen that is expressed or overexpressed in certain solid tumors and identified to be expressed in the solid tumor of the subject to be treated.

The NK cell comprising at least one tumor antigen binding cell surface receptor or the CAR-NK cell may further comprise a CAR containing NKG2D fused to the signaling domain of CD3 and further expressing DAP10. NKG2D is a C-type lectin-like receptor. Human NKG2D receptor monomers assemble into a hexameric structure via association of the transmembrane domain with DAP10 dimers. DAP10 functions as an adaptor protein and transduces the signal after ligand binding to NKG2D. NKG2D ligands are induced-self proteins, which are completely absent or present only at low levels on the surface of normal cells, but are overexpressed, e.g., by transformed cells (tumor antigen). Thus, the CAR may contain an extracellular domain and a transmembrane domain from a natural receptor fused to a CD3 signaling domain and further associating to DAP10. As the NKG2D receptor recognizes several different ligands, which are frequently upregulated during cellular stress, NKG2D-CAR-NK cells, are also multispecific and may be less prone to antigen-loss of the tumor cells. This receptor, although developed for CAR-NK cells is also suitable for CAR-T cells and CAR-NKT cells.

According to the invention engineered NKT cells or NK cells comprising at least one tumor antigen binding protein on its cell surface also include “armored CAR-NK cells” or “armored CAR-NKT cells”. Armored CAR-NK cells have been further optimized to inducibly or constitutively secrete active cytokines or express ligands that further armor the CAR cells to improve efficacy and persistence. The choice of the “armor” agent is based on the knowledge of the tumor microenvironment and the roles of other elements of the innate and adaptive immune system. Examples are interleukins, such as IL-2 and IL-15 for NK cells and IL-15 for NKT cells. These agents have been shown to further enhance CAR-NKT cell and CAR-NK cell efficacy and persistence in tumor microenvironment via different mechanisms. These are typically expressed as an independent gene in the same CAR vector.

NK cells do not persist after adoptive transfer without cytokine support. While the shorter life-span of NK cells may be advantageous, allowing for antitumor activity while reducing the probability of long-term adverse events, such as prolonged cytopenia caused by on-target/off-tumor toxicity to normal tissue, it may also limit their efficacy. For in vivo survival and proliferation, NK cells require continuous cytokine support, without which they are detectable in the circulation for only 1-2 weeks. The two most commonly used cytokines to support persistence of adoptively transferred NK cells are IL-2 and IL-15. Thus, genes for IL-2 and/or IL-15 may be incorporated within the CAR construct of the CAR-NK cell. This allows for constantly providing cytokine support to the CAR-transduced cell.

Interleukins may also be provided exogenously, however, infusion of IL-2 or IL-15 has substantial side-effects. An alternative or additional approach to exogenous administration of cytokines is lymphodepletion chemotherapy prior to adoptive cell transfer of NK cells, such as cyclophosphamide and fludarabine. This provides a favorable environment for NK cell expansion by depleting mature lymphocytes (which consume IL-15), resulting in a marked increase in endogenous IL-15 levels. Apart from the choice of target epitope, the CAR design and the dosing and administration regiment applied, for successful CAR-T cell, CAR-NKT cell or CAR-NK cell therapy of solid tumors, efficient tumor homing and long-term survival in the tumor environment are also important. In most cases the subject is lymphodepleted prior to the administration of CAR-T cells, CAR-NKT cells or CAR-NK cells and potential subsequent cytokine support is further important.

Common side effects of CAR-T cells in liquid tumors is the abundant production of cytokines that may result in a severe cytokine release syndrome (CRS). This risk is less pronounced for solid tumors. Without being bound by theory, this may be explained by a different effector-target cell stoichiometry, which is typically higher in liquid tumors compared to solid tumors. Furthermore, this risk is generally believed to be less for adoptive cell transfer of NKT cells or NK cells compared to T cells, particularly conventional αβT cells.

Furthermore, most tumor antigens are not tumor selective (tumor specific antigens), particularly in solid tumors, and are often merely overexpressed (tumor associated antigens). Thus, there is a risk of off-tumor toxicity. However, ways to reduce off-tumor toxicity are known in the art. For example, off-tumor toxicity may be controlled by administering the required numbers of cells at two, three or more doses. Also RNA transiently-engineered CAR-T cells, CAR-NKT cells or CAR-NK cells may be used to minimize off-tumor toxicity. Also starting the treatment at the earlier stages of tumor development, before the number of cancer cells become too high is beneficial for the outcome and to reduce off-tumor toxicity.

Furthermore target selectivity may be ensured by recognition of two tumor antigens expressed on the same cell. This may be achieved using tandem CARs mediating bispecific activation of T cells, NKT cells or NK cells through the engagement of two chimeric receptors designed to deliver stimulatory and costimulatory signals, such as CD3 ζ-chain signaling domain and CD28 costimulatory domain, in separate CARs, requiring the independent engagement of two different tumor antigens for efficient signaling. Also inhibitory chimeric antigen receptors (iCAR) may be used to divert CAR-T cell, CAR-NKT cell or CAR-NK cell activity from normal tissue. iCARs bind native antigen (i.e., presented on normal tissue only) and comprise a suppressive signaling domain, such as from PD-1 or CTLA-4, to shut down the activation of an active CAR. Thus both approaches, tandem CAR and iCAR, combine the activity of two chimeric antigen receptors.

One problem associated particularly with CAR-T cell immunotherapy, but also with CAR-NKT cell or CAR-NK cell immunotherapy, is the antigen escape that may render CAR-T cells, CAR-NKT cells or CAR-NK cells inefficient against cancer cells. Furthermore, CAR-T cells, CAR-NKT cells or CAR-NK cells are typically not administered repeatedly. Thus, there is a need for a follow-up therapy that allows targeting other tumor antigens, preferably multiple tumor antigens, such as neoantigens.

For safety reasons CAR-modified T cells and possibly also NKT and NK cells may further contain a suicide system, such as inducible caspase-9 (iCasp9) or truncated epidermal growth factor receptor (EGFR that that lacks the signaling domain and can be targeted with an anti-EGFR antibody for rapid elimination of the transgenic cell). This may be particularly relevant for CAR-T and CAR-NKT cells. While mature CAR-NK cells have a limited persistence, NK cells derived from cord blood or hematopoietic stem cells have a higher risk for long-term toxicity and hence suicide systems may also be utilized in these cells.

The inventors have found that the Salmonella typhi Ty21a strain encoding at least one polypeptide comprising five or more neoantigens is particularly suitable as follow-up therapy, because it allows targeting multiple neoantigens expressed in a solid tumor. The Salmonella typhi Ty21a strain encoding at least one polypeptide comprising five or more neoantigens is therefore administered following adoptive cell transfer of the at least one engineered T cell, NKT cell or NK cell comprising at least one tumor antigen binding cell surface receptor. In case the subject has not undergone lymphodepleting chemotherapy prior to adoptive cell transfer of the at least one engineered T cell, NKT cell or NK cell comprising at least one tumor antigen binding cell surface receptor, the Salmonella typhi Ty21a strain encoding at least one polypeptide comprising five or more neoantigens may also be administered together or shortly after (e.g., within a few hours or days) the adoptive cell transfer of the at least one engineered T cell, NKT cell or NK cell comprising at least one tumor antigen binding cell surface receptor.

The at least one engineered T cell, NKT cell or NK cell comprising at least one tumor antigen binding cell surface receptor is typically administered in one adoptive cell transfer and is not repeatedly administered. However the required number of engineered T cells, NKT cells or NK cells may be administered as split doses in two or three subsequent adoptive cell transfers. The treatment may therefore involve a first and optionally a second, a third possibly even further administration within two or more days. Persistence of adoptively transferred cells may be up to three months, up to four months or up to six months. Some authors even claim them to be living cells with a life-long presence.

Prior to the adoptive cell transfer of the at least one engineered T cell, NKT cell or NK cell comprising at least one tumor antigen binding cell surface receptor, the subject may be treated with Salmonella typhi Ty21a comprising a DNA molecule comprising at least one eukaryotic expression cassette encoding VEGFR-2 (e.g., as disclosed in WO 2014/005683). In one embodiment the VEGFR-2 comprises the amino acid sequence of SEQ ID NO: 1. This vaccine (VXM01) is known to enhance the number of tumor infiltrating lymphocytes (TIL). Thus, this vaccine may enhance the efficacy of the at least one engineered T cell, NKT cell or NK cell comprising at least one tumor antigen binding cell surface receptor. Furthermore, since the at least one engineered T cell, NKT cell or NK cell comprising at least one tumor antigen binding cell surface receptor is typically manufactured on-demand, this vaccine provides cancer immunotherapy while the at least one engineered T-cell, NKT cell or NK cell comprising at least one tumor antigen binding cell surface receptor is prepared. Thus, in a specific embodiment the subject is first treated with Salmonella typhi Ty21a comprising a DNA molecule comprising at least one eukaryotic expression cassette encoding VEGFR-2, followed by adoptive cell transfer of the at least one engineered T cell, NKT cell or NK cell comprising at least one tumor antigen binding cell surface receptor (with or without lymphodepleting chemotherapy) and the Salmonella typhi Ty21a encoding at least one polypeptide comprising five or more neoantigens.

In certain embodiments, the Salmonella typhi Ty21a strain encoding at least one polypeptide comprising five or more neoantigens is to be administered at the same time or about two weeks to 4 months, preferably 2 to 3 months, after a first adoptive cell transfer of the at least one engineered T cell, NKT cell or NK cell comprising at least one tumor antigen binding cell surface receptor. Wherein at the same time means within a few hours or days, preferably at the same day or within a week.

In certain embodiments the subject has undergone lymphodepletion, particularly lymphodepleting chemotherapy, prior to adoptive cell transfer of the at least one engineered T cell, NKT cell or NK cell comprising at least one tumor antigen binding cell surface receptor. The term “lymphodepleting chemotherapy” as used herein refers to a chemotherapy that results in lymphophenia in the subject prior to adoptive cell transfer. It also includes non-myeloablative lymphodepleting chemotherapy, which may also improve the efficacy of adoptive cell transfer therapies. Lymphodepleting chemotherapy may also be referred to as “conditioning”. Lymphodepleting chemotherapies are known in the art and may involve the use of cyclophosphamide (CTX) or CXT and fludarabine, e.g., for 7 days. CTX does not affect early hematopoietic bone marrow precursors.

In order to elicit an immune response against the neoantigens in a subject having received lymphodepleting chemotherapy prior to adoptive cell transfer, the lymphocytes need to be replenished, or in other words the subject needs to have regained immune competence, before the Salmonella typhi Ty21a strain comprising a DNA molecule comprising at least one eukaryotic expression cassette encoding at least one polypeptide comprising five or more neoantigens is to be administered. Depending on the type of lymphodepleting chemotherapy, lymphocytes are replenished about 1 month to 2 months following lymphodepleting chemotherapy. Typically lymphocytes are replenished about two weeks to 16 weeks, 4 weeks to 16 weeks, 2 weeks to 16 weeks, or 8 weeks to 12 weeks following lymphodepleting chemotherapy. Preferably the Salmonella typhi Ty21a strain comprising a DNA molecule comprising at least one eukaryotic expression cassette encoding at least one polypeptide comprising five or more neoantigens is administered in a subject after the lymphocyte counts have normalized following lymphodepleting chemotherapy, more preferably after the leukocyte counts have normalized following lymphodepleting chemotherapy, more preferably after the subject regained immune competence. Normal lymphocyte counts are within a range of 1000/mm³ and above. Normal leukocyte counts are within a range of 4000/mm³ and above. Immune competence may be determined based on a leukocyte count of 2000/mm³ and above.

In case the subject received lymphodepletion prior to adoptive cell transfer, the Salmonella typhi Ty21a strain encoding at least one polypeptide comprising five or more neoantigens is to be administered about two weeks to 4 months, preferably 1 to 4 months, preferably 2 to 4 months, more preferably 2 to 3 months, after a first adoptive cell transfer of the at least one engineered T cell, NKT cell or NK cell comprising at least one tumor antigen binding cell surface receptor.

Combination Therapies with the Neoantigen Cancer Vaccine

According to the invention, the Salmonella typhi Ty21a strain comprising a DNA molecule comprising at least one eukaryotic expression cassette encoding at least one polypeptide comprising five or more neoantigens may further be co-administered with at least one checkpoint inhibitor. The term “checkpoint inhibitor” is used synonymous with “immune checkpoint inhibitor” herein. Typically checkpoint therapy blocks inhibitory checkpoints, restoring immune system function. Specifically the at least one checkpoint inhibitor may be an antibody, particularly selected from a group consisting of antibodies against PD-1, PD-L1, CTLA-4, IDO, GITR, OX40, TIM-3, LAG-3, KIR, CSF1R and CD137. The checkpoint inhibitor may be administered simultaneously or separately with the at least one Salmonella typhi Ty21a strain comprising a DNA molecule comprising at least one eukaryotic expression cassette encoding at least one polypeptide comprising five or more neoantigens.

The at least one checkpoint inhibitor, is preferably administered in the approved galenic formulation of the commercial product.

In the context of the present invention, the term “simultaneously” means administration of the attenuated strains of Salmonella typhi Ty21a comprising at least one eukaryotic expression cassette encoding at least one polypeptide comprising five or more neoantigens and the checkpoint inhibitor on the same day, more particularly within 12 hours, more particularly within 2 hours. The term “separately” as used in this context means administration at different days, more particularly at different administration regimens, and in different dosage forms.

The Salmonella typhi Ty21a strain encoding at least one polypeptide comprising five or more neoantigens together with at least one checkpoint inhibitor in a subject that has been or is treated with at least one engineered T cell, NKT cell or NK cell comprising at least one tumor antigen binding cell surface receptor surprisingly show synergistic effects on T cell, NKT cell or NK cell responses and/or overall survival at relatively low doses of the Salmonella typhi Ty21a strain encoding at least one polypeptide comprising five or more neoantigens. Administration of low doses of live bacterial vaccines minimizes the risk of excretion and thus of transmission to third parties.

According to the invention, the subject receiving the Salmonella typhi Ty21a strain comprising a DNA molecule comprising at least one eukaryotic expression cassette encoding at least one polypeptide comprising five or more neoantigens may further be treated with at least one Salmonella typhi Ty21a comprising a DNA molecule comprising at least one eukaryotic expression cassette encoding at least one tumor antigen, tumor stroma antigen and/or checkpoint inhibitor antigen. In one embodiment the at least one tumor antigen, tumor stroma antigen and/or checkpoint inhibitor antigen is selected from the group consisting of human Wilms' Tumor protein (WT1), human Mesothelin (MSLN), human CEA, CMV pp65, human PD-L1, human VEGFR-2 and human fibroblast activation protein (FAP), preferably selected from human PD-L1 and human VEGFR-2. Particularly wherein the treatment further comprises at least one Salmonella typhi Ty21a comprising a DNA molecule comprising at least one eukaryotic expression cassette encoding an antigen selected from the group consisting of WT1, MSLN, CEA, CMV pp65, PD-L1, VEGFR-2 and FAP to be administered to the subject. The at least one Salmonella typhi Ty21a comprising a DNA molecule comprising at least one eukaryotic expression cassette encoding an antigen selected from the group consisting of WT1, MSLN, CEA, CMV pp65, PD-L1, VEGFR-2 and FAP may be administered simultaneously or separately with the at least one Salmonella typhi Ty21a comprising a DNA molecule comprising at least one eukaryotic expression cassette encoding at least one polypeptide comprising five or more neoantigens.

In the context of the present invention, the term “simultaneously” means administration of the different attenuated strains of Salmonella typhi Ty21a on the same day, more particularly within 12 hours, more particularly within 2 hours. The different attenuated strains of Salmonella typhi Ty21a may be, but do not need to be, in the same dosage form. The term “separately” as used in this context means administration at different days, more particularly at different administration regimens, and in different dosage forms.

In particular embodiments human VEGFR-2 comprises the amino acid sequence of SEQ ID NO: 1 or an amino acid sequence that has at least 80% sequence identity with the amino acid sequence of SEQ ID NO: 1. In particular embodiments human Wilms' Tumor Protein (WT1) comprises the amino acid sequence of SEQ ID NO: 3 or an amino acid sequence that has at least 80% sequence identity with the amino acid sequence of SEQ ID NO: 3. In particular embodiments human Mesothelin (MSLN) comprises the amino acid sequence of SEQ ID NO: 4 or an amino acid sequence that has at least 80% sequence identity with the amino acid sequence of SEQ ID NO: 4. In particular embodiments human CEA comprises the amino acid sequence of SEQ ID NO: 5 or an amino acid sequence that has at least 80% sequence identity with the amino acid sequence of SEQ ID NO: 5. In particular embodiments CMV pp65 comprises the amino acid sequence of SEQ ID NO: 6, 7 or 8 or an amino acid sequence that has at least 80% sequence identity with the amino acid sequence of SEQ ID NO: 6, 7 or 8. In particular embodiments human PD-L1 comprising the amino acid sequence of SEQ ID NO: 9 or 10, or an amino acid sequence that has at least 80% sequence identity with the amino acid sequence of SEQ ID NO: 9, 10 or 11.

Preferably VEGFR-2 has the amino acid sequence of SEQ ID NO: 1, WT1 has the amino acid sequence of SEQ ID NO: 3, MSLN has the amino acid sequence of SEQ ID NO: 4, CEA has the amino acid sequence of SEQ ID NO: 5, CMV pp65 has the amino acid sequence of SEQ ID NO: 6, 7 or 8 and/or PD-L1 has the amino acid sequence of SEQ ID NO: 9, 10 or 11.

VEGFR-2, also known as kinase-insert-domain-containing receptor (KDR), appears to mediate almost all of the known cellular responses to VEGF. For example, the role of VEGF in angiogenesis appears to be mediated through the interaction of this protein with VEGFR-2. VEGFR-2 is a 1356 amino acid long, 200-230 kDa molecular weight high-affinity receptor for VEGF, as well as for VEGF-C and VEGF-D. Identified in humans through the screening of endothelial cDNA for tyrosine kinase receptors, VEGFR-2 shares 85% sequence identity with the previously discovered mouse fetal liver kinase 1 (Flk-1). VEGFR-2 is normally expressed in endothelial and hematopoietic precursors, as well as in endothelial cells, nascent hematopoietic stem cells and the umbilical cord stroma. However, in quiescent adult vasculature, VEGFR-2 mRNA appears to be down regulated.

The extracellular domain of VEGFR-2 contains 18 potential N-linked glycosylation sites. VEGFR-2 is initially synthesized as a 150 kDa protein and rapidly glycosylated to a 200 kDa intermediate form, and then further glycosylated at a slower rate to a mature 230 kDa protein which is expressed on the cell surface.

Mesothelin is a 40-kDa cell surface glycoprotein present on normal mesothelial cells and overexpressed in several human tumors, including mesothelioma and ovarian and pancreatic adenocarcinoma. The mesothelin gene encodes a precursor protein of 71-kDa that is processed to yield a 31-kDa shed protein named megakaryocyte-potentiating factor (MPF) and the 40-kDa cell bound fragment mesothelin. Mesothelin was shown to exhibit megakaryocyte-colony-forming activity in the presence of interleukin-3. Mesothelin is a tumor differentiation antigen present at low levels on a restricted set of normal adult tissues, such as mesothelium, but aberrantly overexpressed in a wide variety of human tumors including mesotheliomas, ovarian and pancreatic cancers, squamous cell carcinomas of the cervix, head and neck, vulva, lung and esophagus, lung adenocarcinomas, endometrial carcinomas, biphasic synovial sarcomas, desmoplastic small round cell tumors and gastric adenocarcinomas. The normal biological function of Mesothelin is unknown. Studies in mesothelin knock-out mice revealed no detectable phenotype, and both male and female mice produced healthy off-spring. Studies in pancreatic cancer suggest that mesothelin plays a role in tumorigenesis by increasing cellular proliferation, migration, and S-phase cell populations. Furthermore, there is evidence that mesothelin is an immunogenic protein. Due to its expression profile, its oncogenic functions and its immunogenic potential, the tumor antigen mesothelin is a promising candidate for the development of cancer vaccines.

Wilms' tumor gene 1 (WT1) encodes a zinc finger transcription factor involved in cell proliferation and differentiation. The WT1 protein contains four zinc finger motifs at the C-terminus and a proline/glutamine-rich DNA-binding domain at the N-terminus. Multiple transcript variants, resulting from alternative splicing at two coding exons, have been well characterized. WT1 plays an essential role in the development of the urogenital system and is involved in cell proliferation and differentiation. The WT1 gene was isolated as the gene responsible for a childhood renal neoplasm, Wilms' tumor. It is highly expressed in a wide variety of malignancies including several types of hematological malignancies and various solid tumors. In contrast, normal tissue expression of WT1 in adults is restricted to gonads, uterus, kidney, mesothelium and progenitor cells in various types of tissues. WT-1 negatively affects differentiation and promotes proliferation of progenitor cells. Furthermore, overexpressed WT1 is immunogenic; WT1 specific T-cells as well as IgG anti-WT1 antibodies have been observed in cancer patients. Due to its expression profile, its oncogenic functions and its immunogenic potential, the tumor antigen WT1 is a promising candidate for the development of cancer vaccines. In particular embodiments, WT1 is truncated. In particular embodiments, the zinc finger domain of WT1 is deleted. In particular embodiments, the truncated WT1 has the amino acid sequence as found in SEQ ID NO 3.

The zinc finger domain at the C-terminus of WT1 comprises four zinc finger motifs. Truncated WT1 of the amino acid sequence as found in SEQ ID NO 3 represents amino acids 1 to 371 of UniProt ref P19544-7. Deletion of the zinc finger domain minimizes the risk of immunological cross reactivity with other zinc finger containing transcription factors. Furthermore, truncated WT1 lacking the zinc finger domain has greater immunogenic potential than full-length WT1. In addition, deletion of the zinc finger motifs, which are essential for DNA binding, abrogates the oncogenic potential of WT1, thus minimizing the risk of oncogenesis.

The tegument protein CMV pp65 is a major immunodominant protein of human cytomegalovirus (CMV). The biologic function of CMV pp65 is unclear, but it is believed to be involved in cell cycle regulation. CMV pp65 is a nucleotropic protein exhibiting protein kinase activity, which is able to bind polo-like kinase 1 (PLK-1). HCMV pp65 is expressed in more than 90% of glioblastoma specimens but not in surrounding normal brain. This viral protein is thus a promising candidate as tumor-specific target for the development novel of cancer immunotherapies.

The CMV pp65 protein contains two bipartite nuclear localization signals (NLSs) at amino acids 415 to 438 and amino acids 537 to 561 near the carboxy terminus and a phosphate binding site related to its kinase activity at lysine-436. Mutating the lysine at position 436 to asparagine and deletion of amino acids 537 to 561 results in a protein without kinase activity and markedly reduced nuclear localization. This mutant protein exhibits unaltered immunogenicity.

In particular embodiments, the CMV pp65 has the amino acid sequence as found in SEQ ID NO 6. SEQ ID NO 6 represents the amino acid sequence of wild type CMV pp65. In particular other embodiments, the CMV pp65 has the amino acid sequence as found in SEQ ID NO 7. SEQ ID NO 7 represents the amino acid sequence of CMV pp65, which harbors the mutation K436N relative to the wild type human CMV pp65 of SEQ ID NO 6. In particular other embodiments, the CMV pp65 has the amino acid sequence as found in SEQ ID NO 8. SEQ ID NO 8 represents the amino acid sequence of a truncated version of CMV pp65 of SEQ ID NO 7, which lacks the second, more C-terminal NLS (nuclear localization sequence) (i.e. amino acids 537 to 561 of CMV pp65 of SEQ ID NO 7).

Carcinoembryonic antigen (CEA) (also known as CEACAM5 and CD66e) is a member of a family of highly related glycosyl phosphatidyl inositol (GPI) cell surface anchored glycoproteins involved in cell adhesion. CEA is normally produced in gastrointestinal tissue during fetal development; protein expression ends before birth. Therefore CEA is usually present only at very low levels in the blood of healthy adults. However, the serum levels are raised in some types of cancer, in particular colorectal carcinoma, thus serving as tumor marker. CEA levels may also be raised in gastric carcinoma, pancreatic carcinoma, lung carcinoma, breast carcinoma, and medullary thyroid carcinoma, as well as some non-neoplastic conditions like ulcerative colitis, pancreatitis, cirrhosis, COPD, Crohn's disease and hypothyroidism.

Programmed cell death 1 (PD-1) is expressed on the surface of T-cells and transmits inhibitory signals that maintain T-cell functional silence against cognate antigens. Its ligand PD-L1 is normally expressed on antigen-presenting cells, placental cells and non-hematopoietic cells in inflammatory microenvironments. PD-L1 has been reported to be expressed on immunosuppressive myeloid-derived suppressor cells (MDSC). In addition, PD-L1 is extensively expressed on the surface of various types of cancer cells, which use the PD-1/PD-L1 signaling axis to escape the host immune system. Expression of PD-L1 by cancer cells was shown to correlate with disease stage and poor patient prognosis.

In particular embodiments, PD-L1 is selected from the group consisting of full length PD-L1 and a truncated PD-L1 comprising the extracellular domain of PD-L1. A truncated PD-L1 may comprise an amino acid sequence of amino acids 19 to 238 of of SEQ ID NO: 11, the amino acid sequence of SEQ ID NO: 11, the amino acid sequence of SEQ ID NO: 10 or may comprise an amino acid sequence that shares at least 80% sequence identity with amino acids 19 to 238 of SEQ ID NO: 11, with SEQ ID NO: 11 or with SEQ ID NO: 10. In particular embodiments the PD-L1 is selected from the group consisting of PD-L1 having the amino acid sequence as found in SEQ ID NO: 9 and a protein that shares at least 80% sequence identity therewith. In particular other embodiments PD-L1 is selected from the group consisting of PD-L1 having the amino acid sequence as found in SEQ ID NO: 10 and a protein that shares at least 80% sequence identity therewith. In particular other embodiments PD-L1 is selected from the group consisting of PD-L1 having the amino acid sequence as found in SEQ ID NO: 11 and a protein that shares at least 80% sequence identity therewith. In particular other embodiments PD-L1 is selected from the group consisting of PD-L1 having the amino acid sequence of amino acids 19 to 238 of SEQ ID NO: 11 and a protein that shares at least 80% sequence identity therewith. Particularly, PD-L1 has the amino acid sequence as found in SEQ ID NO: 9, SEQ ID NO: 10 or SEQ ID NO: 11, preferably PD-L1 comprises the amino acid sequence of amino acids 19 to 238 of SEQ ID NO: 11. In one embodiment PD-L1 comprises at least the extracellular domain with or without the signaling peptide.

As used herein, the term “about” or “approximately” means within 80% to 120%, alternatively within 90% to 110%, including within 95% to 105% of a given value or range.

In the context of the present invention, the term “protein that shares at least about 80% sequence identity with the amino acid sequence of SEQ ID NO: X” refers to a protein that has an amino acid sequence with more than 80% amino acid identity when aligned with the amino acid sequence provided. The protein may be of natural origin, e.g. a mutant version of a wild-type protein, e.g. a mutant version of a wild type VEGFR-2 protein, or a homolog of a different species, or an engineered protein, e.g. an engineered VEGFR-2 protein. Methods for designing and constructing derivatives of a given protein are well known to anyone of ordinary skill in the art.

The protein that shares at least about 80% sequence identity with a given amino acid sequence may contain one or more mutations comprising an addition, a deletion and/or a substitution of one or more amino acids in comparison to the reference amino acid sequence. According to the teaching of the present invention, said deleted, added and/or substituted amino acids may be consecutive amino acids or may be interspersed over the length of the amino acid sequence of the protein that shares at least about 80% sequence identity with a given reference protein. According to the teaching of the present invention, any number of amino acids may be added, deleted, and/or substitutes, as long as the amino acid sequence identity with the reference amino acid sequence is at least about 80% and the mutated protein is immunogenic. Preferably, the immunogenicity of the protein which shares at least about 80% sequence identity with the reference amino acid sequence is reduced by less than 50%, less than 40%, less than 30%, less than 20%, less than 10%, less than 5% or less than 1% compared to the reference amino acid sequence, as measured by ELISA. Methods for designing and constructing protein homologues and for testing such homologues for their immunogenic potential are well known to anyone of ordinary skill in the art. In particular embodiments, the sequence identity with the reference amino acid is at least about 85%, at least about 90%, at least about 95% and most particularly at least about 99%. Methods and algorithms for determining sequence identity including the comparison of a parental protein and its derivative having deletions, additions and/or substitutions relative to a parental sequence, are well known to the practitioner of ordinary skill in the art. On the DNA level, the nucleic acid sequences encoding the protein that shares at least about 80% sequence identity with the reference amino acid sequence may differ to a larger extent due to the degeneracy of the genetic code.

In particular embodiments, the administration of the Salmonella typhi Ty21a strain encoding at least one polypeptide comprising five or more neoantigens is combined with the administration of the attenuated strains of Salmonella encoding a tumor antigen or tumor stroma antigen selected from WT1, MSLN, CEA, CMV pp65, PD-L1, VEGFR-2 and FAP and one checkpoint inhibitor.

In particular embodiments, the treatment may also be accompanied by chemotherapy or radiotherapy. For cure of cancer, complete eradication of cancer stem cells may be essential. For maximal efficacy, a combination of different therapy approaches may therefore be beneficial.

Chemotherapeutic agents that may be used in combination with the Salmonella typhi Ty21a strain of the present invention may be, for example: gemcitabine, amifostine (ethyol), cabazitaxel, cisplatin, dacarbazine (DTIC), dactinomycin, docetaxel, mechlorethamine, streptozocin, cyclophosphamide, carrnustine (BCNU), lomustine (CCNU), doxorubicin (adriamycin), doxorubicin lipo (doxil), folinic acid, gemcitabine (gemzar), daunorubicin, daunorubicin lipo (daunoxome), procarbazine, ketokonazole, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, oxaliplatin, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

Most preferred chemotherapeutic agents according to the invention are cabazitaxel, carboplatin, oxaliplatin, cisplatin, cyclophosphamide, docetaxel, gemcitabine, doxorubicin, paclitaxel (taxol), irinotecan, vincristine, vinblastine, vinorelbin, folinic acid, 5-fluorouracil and bleomycin, especially gemcitabine.

Particularly, the Salmonella typhi Ty21a strain is administered before or during the chemotherapy or the radiotherapy treatment. In other particular embodiments, the Salmonella typhi Ty21a strain is administered before and during the chemotherapy or the radiotherapy treatment.

Salmonella typhi Ty21a

Attenuated strains of Salmonella, particularly of the species Salmonella enterica, are attractive vehicles for the delivery of heterologous antigens to the mammalian immune system, since S. enterica strains can potentially be delivered via mucosal routes of immunization, i.e. orally or nasally, which offers advantages of simplicity and safety compared to parenteral administration. Furthermore, Salmonella strains elicit strong humoral and cellular immune responses at the level of both systemic and mucosal compartments. Batch preparation costs are low and formulations of live bacterial vaccines are highly stable. Attenuation can be accomplished by deletion of various genes, including virulence, regulatory, and metabolic genes.

Several Salmonella typhimurium strains attenuated by aro mutations have been shown to be safe and effective delivery vehicles for heterologous antigens in animal models.

According to the invention, the attenuated strain of Salmonella is Salmonelly enterica serovar typhi strain Ty21a, also referred to as Salmonella typhi Ty21a. The live, attenuated S. typhi Ty21a strain is the active component of Typhoral L®, also known as Vivotif® (manufactured by Berna Biotech Ltd., a Crucell Company, Switzerland). It is currently the only licensed live oral vaccine against typhoid fever. This vaccine has been extensively tested and has proved to be safe regarding patient toxicity as well as transmission to third parties (Wandan et al., J. Infectious Diseases 1982, 145:292-295). The vaccine is licensed in more than 40 countries and has been used in millions of individuals including thousands of children for prophylactic vaccination against typhoid fever. It has an unparalleled safety track record. There is no data available indicating that S. typhi Ty21a is able to enter the bloodstream systemically. The live attenuated Salmonella typhi Ty21a vaccine strain thus allows specific targeting of the immune system in the gut, while being safe and well-tolerated. The Marketing Authorization number of Typhoral Le is PL 15747/0001 dated 16 Dec. 1996. One dose of vaccine contains at least 2×10⁹ viable S. typhi Ty21a colony forming units and at least 5×10⁹ non-viable S. typhi Ty21a cells.

This well-tolerated, live oral vaccine against typhoid fever was derived by chemical mutagenesis of the wild-type virulent bacterial isolate S. typhi Ty2 and harbors a loss-of-function mutation in the galE gene resulting in its inability to metabolize galactose. The attenuated bacterial strain is also not able to reduce sulfate to sulfide which differentiates it from the wild-type Salmonella typhi Ty2 strain. With regard to its serological characteristics, the Salmonella typhi Ty21a strain contains the 09-antigen which is a polysaccharide of the outer membrane of the bacteria and lacks the 05-antigen which is in turn a characteristic component of Salmonella typhimurium. This serological characteristic supports the rationale for including the respective test in a panel of identity tests for batch release.

The expression cassette as used in the Salmonella typhi Ty21a strain according to the invention is a eukaryotic expression cassette, particularly comprising a CMV promoter. In the context of the present invention, the term “eukaryotic expression cassette” refers to an expression cassette which allows for expression of the open reading frame in a eukaryotic cell. It has been shown that the amount of heterologous antigen required to induce an adequate immune response may be toxic for the bacterium and may result in cell death, over-attenuation or loss of expression of the heterologous antigen. Using a eukaryotic expression cassette that is not expressed in the bacterial vector but only in the target cell may overcome this toxicity problem and the protein expressed typically exhibits a eukaryotic glycosylation pattern.

A eukaryotic expression cassette comprises regulatory sequences that are able to control the expression of an open reading frame in a eukaryotic cell, preferably a promoter and a polyadenylation signal. Promoters and polyadenylation signals included in the eukaryotic expression cassette comprised by the Salmonella typhi Ty21a strain of the present invention are preferably selected to be functional within the cells of the subject to be immunized. Examples of suitable promoters, especially for the production of a DNA vaccine for humans, include but are not limited to promoters from Cytomegalovirus (CMV), such as the strong CMV immediate early promoter, Simian Virus 40 (SV40), Mouse Mammary Tumor Virus (MMTV), Human Immunodeficiency Virus (HIV), such as the HIV Long Terminal Repeat (LTR) promoter, Moloney virus, Epstein Barr Virus (EBV), and from Rous Sarcoma Virus (RSV), the synthetic CAG promoter composed of the CMV early enhancer element, the promoter, the first exon and the first intron of chicken beta-actin gene and the splice acceptor of the rabbit beta globin gene, as well as promoters from human genes such as human actin, human myosin, human hemoglobin, human muscle creatine, and human metallothionein. In a particular embodiment, the eukaryotic expression cassette contains the CMV promoter. In the context of the present invention, the term “CMV promoter” refers to the strong immediate-early cytomegalovirus promoter.

Examples of suitable polyadenylation signals, especially for the production of a DNA vaccine for humans, include but are not limited to the bovine growth hormone (BGH) polyadenylation site, SV40 polyadenylation signals and LTR polyadenylation signals. In a particular embodiment, the eukaryotic expression cassette comprised by the Salmonella typhi Ty21a strain of the present invention comprises the BGH polyadenylation site.

In addition to the regulatory elements required for expression of the heterologous polypeptide, like a promoter and a polyadenylation signal, other elements can also be included in the eukaryotic expression cassette. Such additional elements include enhancers. The enhancer can be, for example, the enhancer of human actin, human myosin, human hemoglobin, human muscle creatine and viral enhancers such as those from CMV, RSV and EBV.

Regulatory sequences and codons are generally species dependent, so in order to maximize protein production, the regulatory sequences and codons are preferably selected to be effective in the species to be immunized. The person skilled in the art can produce recombinant DNA molecules that are functional in a given subject species, as for example a human subject.

In particular embodiments, the DNA molecule or the DNA molecule comprising the at least one eukaryotic expression cassette comprise an antibiotic resistance gene, such as the kanamycin antibiotic resistance gene, an ori, such as the pMB1 ori or the pUC, and a strong promoter, such as a CMV promoter. In particular embodiments, the recombinant DNA molecule or the DNA molecule comprising the at least one eukaryotic expression cassette is a plasmid, such as a plasmid based on or derived from the commercially available pVAX1™ expression plasmid (Invitrogen, San Diego, Calif.).

This expression vector may be modified by replacing the high copy pUC origin of replication by the low copy pMB1 origin of replication of pBR322. The low copy modification was made in order to reduce the metabolic burden and to render the construct more stable. The generated expression vector backbone was designated pVAX10.

In particular embodiments, the expression plasmid comprises the DNA molecule of SEQ ID NO: 2 (vector backbone pVAX10), which correlates to the sequence of expression vector pVAX10 without the portion of the multiple cloning site which is located between the restriction sites NheI and XhoI.

In particular embodiments, the Salmonella typhi Ty21a strain is administered orally. Oral administration is simpler, safer and more comfortable than parenteral administration. However, it has to be noted that the Salmonella typhi Ty21 strain of the present invention may also be administered by any other suitable route. Preferably, a therapeutically effective dose is administered to the subject, and this dose depends on the particular application, the type of malignancy, the subject's weight, age, sex and state of health, the manner of administration and the formulation, etc. Administration may be single or multiple, as required.

The Salmonella typhi Ty21a strain encoding at least one polypeptide comprising five or more neoantigens may be provided in the form of a solution, a suspension, a lyophilisate, an enteric coated capsule, or any other suitable form. Typically, the Salmonella typhi Ty21a strain is formulated as drinking solution. This embodiment offers the advantage of improved patient compliance. Preferably, the drinking solution comprises means to neutralize gastric acids at least to a certain degree, i.e., to bring the pH of the gastric juice closer to a pH of 7. Preferably, the drinking solution is a buffered suspension comprising the Salmonella typhi Ty21a strain according to the present invention. In a particular embodiment, the buffered suspension is obtained by suspending the Salmonella typhi Ty21a strain in a suitable buffer, preferably containing 2.6 g sodium hydrogen carbonate, 1.7 g L-ascorbic acid, 0.2 g lactose monohydrate and 100 ml of drinking water.

In particular embodiments, a single dose of the Salmonella typhi Ty21a strain comprises from about 10⁶ to about 10¹⁰, more particularly from about 10⁶ to about 10⁹, more particularly from about 10⁷ to about 10⁹, more particularly from about 10⁶ to about 10⁸, most particularly from about 10⁶ to about 10⁷ colony forming units (CFU).

More particularly, a single dose of the Salmonella typhi Ty21a strain comprises from about 1×10⁶ to about 1×10¹⁰, more particularly from about 1×10⁶ to about 1×10⁹, more particularly from about 1×10⁷ to about 1×10⁹ more particularly from about 1×10⁶ to about 1×10⁸, most particularly from about 1×10⁶ to about 1×10⁷ colony forming units (CFU).

Furthermore, the Salmonella typhi Ty21a strain according to the invention is preferably administered two to four times in the first week, preferably 4 times in the first week, followed by single dose boosting administration every 2 to 4 weeks, particularly on day 1 and 7, preferably on day 1, 3, 5 and 7 followed by single dose boosting administrations every 2 to 4 weeks.

In this context, the term “about” or “approximately” means within a factor of 3, alternatively within a factor of 2, including within a factor of 1.5 of a given value or range.

In particular embodiments, the treatment comprises a single or multiple administrations of the Salmonella typhi Ty21a strain encoding at least one polypeptide comprising five or more neoantigens or the pharmaceutical composition according to the present invention. The single dose of the administrations may be the same or different, preferably within the ranges as disclosed herein. In particular, the treatment comprises two to four prime vaccinations in the first week of treatment followed by single dose boosting administrations every two to four weeks of the Salmonella typhi Ty21a strain encoding at least one polypeptide comprising five or more neoantigens or the pharmaceutical composition according to the present invention, preferably wherein the multiple administrations occur within three to six consecutive months.

It may be favorable dependent on the occurrence of possible side effects, to include treatment with antibiotics or anti-inflammatory agents.

Should adverse events occur that resemble hypersensitivity reactions mediated by histamine, leukotrienes, or cytokines, treatment options for fever, anaphylaxis, blood pressure instability, bronchospasm, and dyspnoea are available. Treatment options in case of unwanted T-cell derived auto-aggression are derived from standard treatment schemes in acute and chronic graft vs. host disease applied after stem cell transplantation. Cyclosporin and glucocorticoids are proposed as treatment options.

In the unlikely case of systemic Salmonella typhi Ty21a type infection, appropriate antibiotic therapy is recommended, for example with fluoroquinolones including ciprofloxacin or ofloxacin. Bacterial infections of the gastrointestinal tract are to be treated with respective agents, such as rifaximin.

### Pharmaceutical Compositions

In a further aspect, the present invention relates to a pharmaceutical composition comprising a Salmonella typhi Ty21a strain comprising a DNA molecule comprising at least one eukaryotic expression cassette encoding at least one polypeptide comprising five or more neoantigens.

The pharmaceutical composition of the present invention may be in the form of a solution, a suspension, an enteric coated capsule, a lyophilized powder or any other form suitable for the intended use. The pharmaceutical composition of the present invention may further comprise one or more pharmaceutically acceptable excipients.

In the context of the present invention, the term “excipient” refers to a natural or synthetic substance formulated alongside the active ingredient of a medication. Suitable excipients include antiadherents, binders, coatings, disintegrants, flavors, colors, lubricants, glidants, sorbents, preservatives and sweeteners.

In the context of the present invention, the term “pharmaceutically acceptable” refers to molecular entities and other ingredients of pharmaceutical compositions that are physiologically tolerable and do not typically produce untoward reactions when administered to a mammal (e.g., human). The term “pharmaceutically acceptable” may also mean approved by a regulatory agency of a Federal or a state government or listed in the U.S. Pharmacopeia or other generally recognized pharmacopeia for use in mammals, and, more particularly, in humans.

In particular, suitable drinking solutions comprise means to neutralize gastric acids to at least to a certain degree, i.e. to bring the pH of the gastric juice closer to a pH of 7. In a particular embodiment, the drinking solution is a buffered suspension obtained by suspending the Salmonella typhi Ty21a strain according to the present invention in a suitable buffer, preferably in a buffer that neutralizes gastric acids to at least a certain degree, preferably in a buffer containing 2.6 g sodium hydrogen carbonate, 1.7 g L-ascorbic acid, 0.2 g lactose monohydrate and 100 ml of drinking water.

In particular embodiments, the pharmaceutical composition is for use as a medicament, particularly for use in the treatment of a solid tumor in a subject according to the invention. In particular embodiments, the pharmaceutical composition is for use as a medicament, particularly for use in the treatment of a solid tumor in a subject, wherein the subject has been or is treated with at least one engineered T cell, NKT cell or NK cell comprising at least one tumor antigen binding cell surface receptor or any other uses or methods disclosed herein.

### EXAMPLES

### Example 1: Proof of Concept for Eliciting an Immune Response Using a Salmonella typhi Ty21a Strain Encoding a Polypeptide Comprising Multiple Antigens

Generation of the Salmonella typhi Ty21a Strain Encoding a Polypeptide Comprising Multiple Antigens.

A construct comprising 9 dominant CD8 epitopes (2 epitopes of VEGFR-2 (referred to as KDR2, KDR3), 2 epitopes of MSLN (referred to as MSLN_GSL, MSLN_IQL), 1 epitope of WT-1, 3 epitopes of CEA (referred to as CEA-CSA, CEA_CSV, CEA_LTL) and 1 epitope of OVA) was cloned. The epitopes were selected as model antigens based on literature or based on predicted binding properties. It is important to note that the model epitopes selected for proof of concept were not mutated or neoantigens, but dominant CD8 epitopes from known tumor-associated antigens like mesothelin (MSLN), Wilms tumor 1 (WT-1), carcinoembryonic antigen (CEA), tumor-stroma antigens (VEGFR-2) or ovalbumin (OVA).

Assessment of T Cell Responses Against Multiple Antigens Following Vaccination

In this first-in-animal study, C57BL/6 mice received either VXMNeo1m or VXM empty vector vaccines, at 10¹⁰ CFU/administration, twice within one week (q7dx2) by the oral route (p.o.). As a positive control, a group of mice received a prime vaccination (q7dx2) with VXM06m that encodes for the Wilms' tumor (WT1) whole protein. The negative control group received p.o. the empty vector with the same schedule and dose as the VXMNeo1m vector vaccine (10¹⁰ CFU/administration, q7dx2). At study day 17, i.e. 10 days after the last vaccination, mice were euthanized and the spleen was removed for pentamer analysis by flow cytometry (FC).

TABLE 2

| Group | No. | | | | Treatment |
|---|---|---|---|---|---|
| # | Animals | Vaccine | Dose | Route | Schedule |
| 1 | 5 | empty vector | 1010 CFU/adm | p.o. | dl, d7 |
| 2 | 5 | VXM06m | 1010 CFU/adm | p.o. | dl, d7 |
| 3 | 5 | VXMNeo1m | 1010 CFU/adm | p.o. | dl, d7 |

Test substances were applied by oral gavage via a gavage tube. Regardless of animal groups, each animal received pre-dose application buffer by oral gavage to neutralize acid in the stomach prior dosing (100 μL/animal/application). This buffer was composed by dissolution of 2.6 g sodium hydrogen carbonate, 1.7 g L-Ascorbic acid, 0.2 g lactose monohydrate in 100 mL of drinking water and was applied within 30 min prior application of test substances. The applications were freshly prepared the day of application.

Epitope-specific CD8 T cells were analyzed by flow cytometry on splenocytes collected 10 days after the last vaccination, with a set of specific pentamers. An irrelevant pentamer, i.e. HPV 16 E7 49-57, was used to set the background threshold.

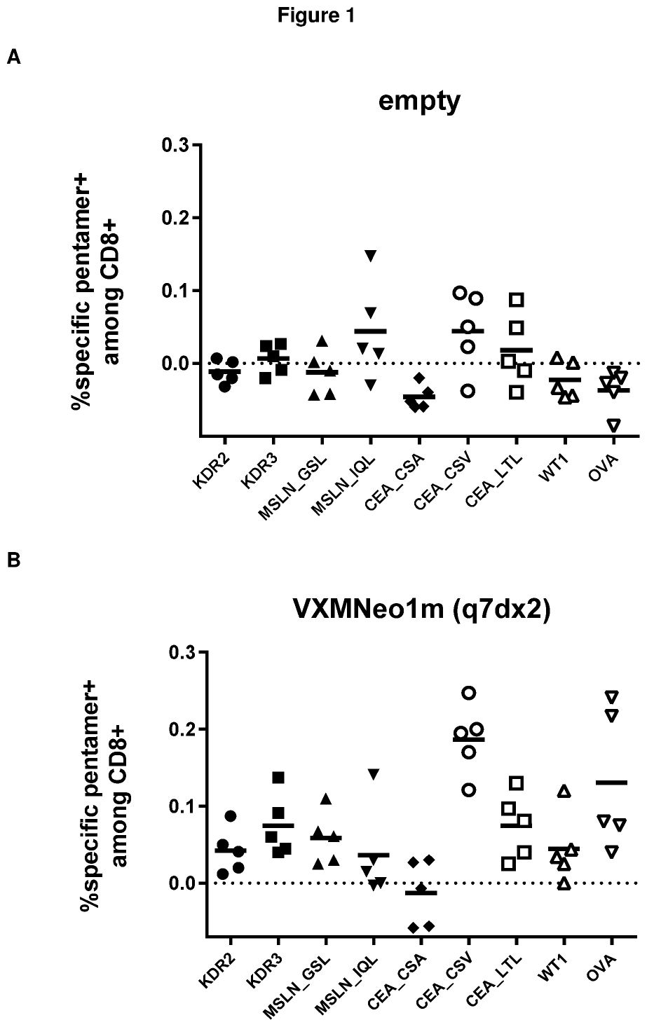

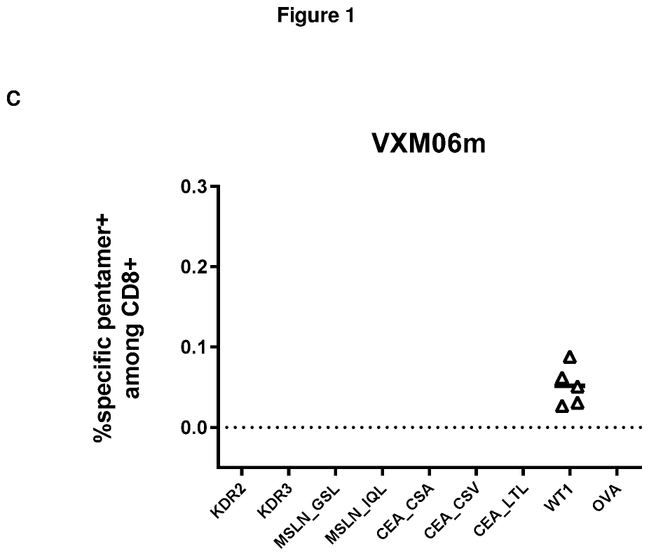

Results of CD8 T cell responses determined using 9 identical peptide pentamer flow cytometry reagents and an additional HPV reagent as negative control are shown in FIG. 1.

### Example 2: Stability Testing of Drug Product

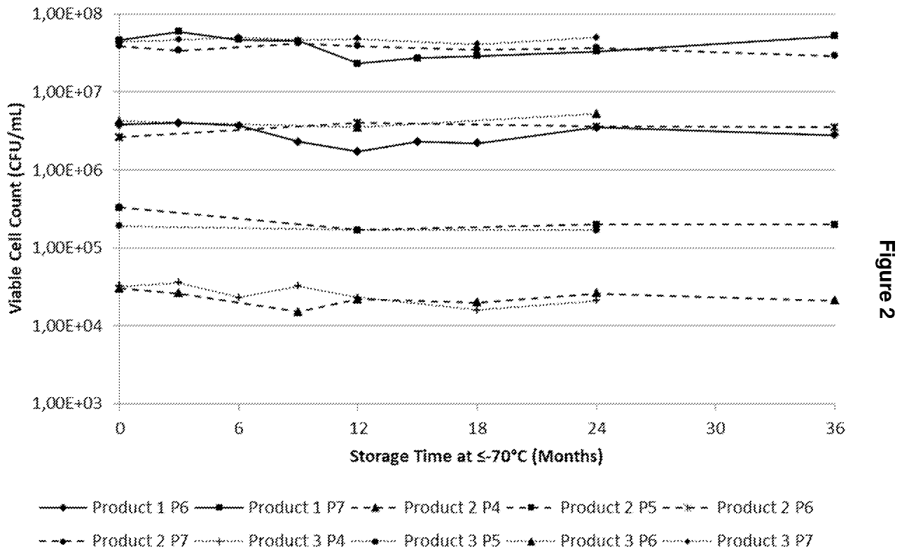

Finished drug products were manufactured from three constructs encoding three different target antigens based on the same Salmonella Typhi Ty21a delivery platform in the identical formulation and container/closure system. Strengths of 10⁴, 10⁵, 10⁶, and 10⁷ CFU/mL with a fill volume of 1.3 mL per vial were manufactured. Vials were placed on stability at ≤−70° C. At different stability time points samples were tested according to a predefined stability protocol for identity, contents, potency, pH, and microbiological purity. Predefined specifications were met at all time points with samples from all three constructs confirming the stability of Vaximm's platform constructs over 3 years when stored at ≤−70° C. irrespective of the cloned insert. Results for testing of viable cell counts are presented in FIG. 2.

### SEQUENCE LISTING

SEQ ID NO: 1 amino acid sequence of human VEGFR-2
SEQ ID NO: 2 nucleotide sequence of expression vector pVAX10 without the multiple cloning site located between the restriction sites NheI and XhoI.
SEQ ID NO: 3 amino acid sequence of truncated (zinc-finger domain deleted) human WT1
SEQ ID NO: 4 amino acid sequence of human MSLN
SEQ ID NO: 5 amino acid sequence of human CEA
SEQ ID NO: 6 amino acid sequence of wildtype CMV pp65
SEQ ID NO: 7 amino acid sequence of CMV pp65 carrying mutation K436N
SEQ ID NO: 8 amino acid sequence of truncated CMV pp65 carrying mutation K436N and lacking the C-terminal NLS (aa 537-561 of SEQ ID NO: 7)
SEQ ID NO: 9 amino acid sequence of human full length human PD-L1
SEQ ID NO: 10 amino acid sequence of human PD-L1 lacking the signaling peptide
SEQ ID NO: 11 amino acid sequence of truncated human PD-L1 comprising the extracellular domain and the signaling peptide

1. A Salmonella typhi Ty21a strain comprising a DNA molecule comprising at least one eukaryotic expression cassette encoding at least one polypeptide comprising five or more neoantigens, for use in the treatment of a solid tumor in a subject, wherein the subject has been or is treated with at least one engineered T cell, NKT cell or NK cell comprising at least one tumor antigen binding cell surface receptor.

2. The Salmonella typhi Ty21a strain for the use according to claim 1 or 2, wherein the five or more neoantigens are tumor specific antigens identified in the solid tumor of said subject.

3. The Salmonella typhi Ty21a strain for the use according to any one of the preceding claims, wherein the five or more neoantigens comprise
(a) CD8 T cell antigens; or
(b) CD8 and CD4 T cell antigens.

4. The Salmonella typhi Ty21a strain for the use according to any one of the preceding claims, wherein the treatment further comprises administration of at least one checkpoint inhibitor.

5. The Salmonella typhi Ty21a strain for the use according to claim 4, wherein the at least one checkpoint inhibitor is selected from a group consisting of antibodies against PD-1, PD-L1, CTLA-4, IDO, GITR, OX40, TIM-3, LAG-3, KIR, CSF1R and CD137.

6. The Salmonella typhi Ty21a strain for the use according to any one of the preceding claims, wherein the at least one engineered T cell, NKT cell or NK cell comprising at least one tumor antigen binding cell surface receptor is a chimeric antigen receptor (CAR)-T cell, a CAR-NKT cell or a CAR-NK cell.

7. The Salmonella typhi Ty21a strain for the use according to any one of the preceding claims, wherein the Salmonella typhi Ty21a strain is to be administered following adoptive cell transfer of the at least one engineered T cell, NKT cell or NK cell comprising at least one tumor antigen binding cell surface receptor.

8. The Salmonella typhi Ty21a strain for the use according to claim 7, wherein the Salmonella typhi Ty21a strain is to be administered
(a) about two weeks to 4 months after a first adoptive cell transfer of the at least one engineered T cell, NKT cell or NK cell comprising at least one tumor antigen binding cell surface receptor; or
(b) about 2 to 3 months after a first adoptive cell transfer of the at least one engineered T cell, NKT cell or NK cell comprising at least one tumor antigen binding cell surface receptor.

9. The Salmonella typhi Ty21a strain for the use according to any one of the preceding claims, wherein the subject has undergone lymphodepleting chemotherapy prior to adoptive cell transfer of the at least one engineered T cell, NKT cell or NK cell comprising at least one tumor antigen binding cell surface receptor.

10. The Salmonella typhi Ty21a strain for the use according to claim 9, wherein lymphocyte and/or leukocyte counts have normalized before the Salmonella typhi Ty21a strain is to be administered.

11. The Salmonella typhi Ty21a strain for the use according to any one of the preceding claims, wherein the Salmonella typhi Ty21a strain is to be administered orally.

12. The Salmonella typhi Ty21a strain for the use according to any one of the preceding claims, wherein the treatment further comprises administration of at least one Salmonella typhi Ty21a strain comprising a DNA molecule comprising at least one eukaryotic expression cassette encoding at least one antigen selected from the group consisting of human Wilms' Tumor protein (WT1), human Mesothelin (MSLN), human CEA, CMV pp65, human PD-L1, VEGFR-2 and human fibroblast activation protein (FAP).

13. The Salmonella typhi Ty21a strain for the use according to any one of the preceding claims, wherein the solid tumor is selected from colorectal cancer, pancreatic cancer, lung cancer, ovarian cancer, mesothelioma, glioblastoma, gastric cancer, hepatocellular cancer, renal cell cancer, prostate cancer, cervical cancer, breast cancer and melanoma.

14. The Salmonella typhi Ty21a strain for the use according to of any one of the preceding claims, wherein
(a) a single dose of the Salmonella typhi Ty21a strain comprises from about 10⁶ to about 10¹⁰, more particularly from about 10⁶ to about 10⁹, more particularly from about 10⁶ to about 10⁸, most particularly from about 10⁷ to about 10⁸ colony forming units (CFU); and/or
(b) wherein the Salmonella typhi Ty21a strain is administered 2 to 4 times in the first week, followed by a single dose boosting administration every 2 to 4 weeks.

15. The Salmonella typhi Ty21a strain for the use according to any one of the preceding claims, wherein the Salmonella typhi Ty21a strain is in the form of a pharmaceutical composition, further comprising at least one pharmaceutically acceptable excipient.


SEQUENCE LISTING (11 sequences)

SEQ ID NO: 1 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 2 - DNA, 3500 bp, Artificial Sequence
  expression plasmid
tgggcttttgctggccttttgctcacatgttcttgactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagacccccatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatc

SEQ ID NO: 3 - protein, 371 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLK

SEQ ID NO: 4 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 5 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 6 - protein, 561 aa, Cytomegalovirus
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 7 - protein, 561 aa, Artificial Sequence
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 8 - protein, 536 aa, Artificial Sequence
  mutated CMV pp65
MESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRNRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQ

SEQ ID NO: 9 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - protein, 273 aa, Homo sapiens
MFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 11 - protein, 238 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER